SEQUENCE LISTING (8 sequences)

SEQ ID NO: 1 - DNA, 16 bp, Artificial Sequence
  Artificial LNA/DNA molecule
  Modified_base (1)..(3): Locked Nucleic Acid (LNA)
  Modified_base (14)..(16): Locked Nucleic Acid (LNA)
ctgtaagcactttgac

SEQ ID NO: 2 - DNA, 15 bp, Artificial Sequence
  Artificial LNA/DNA molecule
  modified_base (1)..(3): Locked Nucleic Acid (LNA)
  modified_base (13)..(15): Locked Nucleic Acid (LNA)
acatcgacacaataa

SEQ ID NO: 3 - DNA, 16 bp, Artificial Sequence
  Artificial LNA/DNA gapmer
  modified_base (1)..(3): Locked Nucleic Acid (LNA)
  modified_base (14)..(16): Locked Nucleic Acid (LNA)
tcagtaacaggacagt

SEQ ID NO: 4 - DNA, 14 bp, Artificial Sequence
  Artificial LNA/DNA gapmer
  modified_base (1)..(3): Locked Nucleic Acid (LNA)
  modified_base (12)..(14): Locked Nucleic Acid (LNA)
tacttgcttggctt

SEQ ID NO: 5 - DNA, 16 bp, Artificial Sequence
  Artificial LNA/DNa gapmer
  modified_base (1)..(3): Locked Nucleic Acid (LNA)
  modified_base (14)..(16): Locked Nucleic Acid (LNA)
atgcaaaactaacaga

SEQ ID NO: 6 - DNA, 16 bp, Artificial Sequence
  Artificial LNA/DNa gapmer
  modified_base (1)..(3): Locked nucleic acid (LNA)
  modified_base (14)..(16): Locked nucleic acid (LNA)
gaaggaaatagcaggc

SEQ ID NO: 7 - DNA, 16 bp, Artificial Sequence
  Artificial LNA/DNA gapmer
  modified_base (1)..(3): Locked Nucleic Acid (LNA)
  modified_base (14)..(16): Locked Nucleic Acid (LNA)
agcactcaacatcagc

SEQ ID NO: 8 - DNA, 15 bp, Artificial sequence
  Artificial LNA/DNA gapmer
  modified_base (1)..(3): Locked Nucleic Acid (LNA)
  modified_base (13)..(15): Locked Nucleic Acid (LNA)
cgacaggccgaagct

(19) United States
(12) Patent Application Publication
(10) Pub. No.: US 2018/0051284 A1
(43) Pub. Date: Feb. 22, 2018

(54) INHIBITORS OF MIR-17-92 CLUSTER FOR ANTI-TUMOR ACTIVITY IN MULTIPLE MYELOMA AND OTHER MALIGNANCIES

(71) Applicants: Pierfrancesco TASSONE, Catanzaro (IT); Pierosandro TAGLIAFERRI, Napoli (IT); Maria Teresa Di Martino, Catanzaro (IT)

(72) Inventors: Pierfrancesco TASSONE, Catanzaro (IT); Pierosandro TAGLIAFERRI, Napoli (IT); Maria Teresa Di Martino, Catanzaro (IT)

(21) Appl. No.: 15/535,018

(22) PCT Filed: Dec. 4, 2015
(86) PCT No.: PCT/EP2015/078655

§ 371 Date: Jun. 9, 2017

(30) Foreign Application Priority Data
Dec. 9, 2014 (IT) RM2014A000712

(51) Int. Cl. C12N 15/113; A61K 31/713; A61K 31/198

(52) U.S. Cl. CPC C12N 15/113; A61K 31/713; A61K 31/198; C12N 2320/31; C12N 2310/3231; C12N 2310/341; C12N 2310/315; C12N 2310/113

(57) ABSTRACT

The present invention refers to inhibitors of the miR-17-92 cluster and to their use as medicaments, in particular in the treatment of multiple myeloma and other malignancies. More in particular, the present invention refers to an LNA/DNA gapmer which binds to a region of the primary miRNA (pri-miRNA) of the miR-17-92 cluster Said inhibitors can be used for the treatment of tumors related to an overexpression of any of the miRNAs of the miR-17-92 cluster. Pharmaceutical compositions are also within the scope of the invention.

### TECHNICAL FIELD

The present invention refers to the field of pharmaceuticals and biotechnology.

In particular, the present invention refers to inhibitors of miR-17-92 cluster, also named miR-17-92-i-PT, and to their use as medicaments, in particular in the treatment of multiple myeloma and other malignancies.

### BACKGROUND OF THE INVENTION

Multiple myeloma (MM) is a hematologic malignancy characterized by proliferation of neoplastic plasma cells in the bone marrow. Although recent therapeutic options for MM have led to a considerable improvement of patient survival, the course of this disease remains lethal in most of cases. A wide number of complex genetic aberrations contributes to the multistep transformation process of plasma cells within the human bone marrow microenvironment (huBMM), which plays an essential role for growth, survival and the drug resistance of tumor cells. There is now a rising body of evidence demonstrating that these aberrations may affect the microRNAs (miRNA) expression in MM, which in turn translate into aberrant translation of messenger RNA. Therefore the miRNA network is progressively disclosing its relevant key involvement in the pathogenesis of this important disease.

MicroRNAs are a class of regulatory non-coding RNAs of 19-25 nucleotides in length that acts by targeting specific messenger RNAs (mRNAs) for degradation or inhibition of translation through base pairing to partially or fully complementary sites. At present, the miRNA network, which includes several hundreds of sequences, is involved in a variety of normal biological functions as well as in tumorigenic events, since deregulated miRNAs can act as oncogenes (Onco-miRNAs) or tumor-suppressors (TS-miRNAs). Changes in gene copy number, chromosomal translocation, mutations, transcriptional activation, epigenetic silencing and defective miRNA development are variably responsible of this deregulation. miRNA mediated post-transcriptional silencing can occur through degradation of target mRNAs (Bagga S, et al. Cell 2005) and/or inhibition of protein synthesis at the initiation stage (Djuranovic S, et al. Science 2012). Due to their small size and imperfect base-pairing with the targets, miRNAs have the capacity to regulate many target mRNAs, therefore acting as global regulators for gene expression.

The biogenesis of these tiny molecules is well known. MiRNA genes reside in regions of the genome as distinct transcriptional unit or in cluster of polycistronic units carrying the information of several miRNAs (Lagos-Quintana et al, 2001, Lee et al 2002). RNA polymerase (RNase) II transcribes miRNA genes generating long primary transcripts, the pri-miRNAs (Kim 2005). In the nucleus, the RNase III enzyme Drosha processes the pri-miRNA yielding a hairpin precursor, the pre-miRNA, consisting of approximately 70 nucleotides (nt). Subsequently, the pre-miRNA hairpins are exported to the cytoplasm by exportin-5 where they are further processed into 19-25 nt miRNA duplex structures by the RNase III Dicer (Sontheimer 2005) to yield mature miRNA duplexes (Kim VN, et al. Nat Rev Mol Cell Biol 2009).

Pri-miRNAs contain either a single hairpin structure or tandem hairpin structures. It turns out that 30% of miRNAs are transcribed as polycistronic miRNA clusters (Megraw M, et al Nucleic Acids Res 2007).

MiRNAs are emerging as new potential multi-target agents, due to their ability to tune the expression of several genes, in the context of signaling networks involved in cancer promotion or repression.

MiR-17-92 cluster gene, also known as “oncomiR-1”, encodes a polycistronic miRNA transcript (the pri-miRNA) that yields six individual miRNA components including miR-17, -18a, -19a, -20a, -19b, and -92a (for review see Mendell J T, Cell 2008). Among miRNAs significantly deregulated in human cancer, those that belong to miR-17-92 cluster retain oncogenic potential. The expression of this cluster of miRNAs is frequently upregulated in both solid and hematologic malignancies, mainly due to genomic amplification and transcriptional activation (Lu J, et al. Nature 2005; He L, et al nature 2005; Hayashita Y et al Cancer Res 2005; Takakura S, Cancer Sci. 2008).

The gene coding for miR-17-92 cluster (MIR17HG) is located at chromosome 13q31, a region amplified in Burkitt's lymphoma, diffuse large B-cell lymphoma, follicular lymphoma, mantle cell lymphoma (Ota A, et al Cancer Res 2004; agawa and Seto, leukemia 2005), breast, lung, renal and colon cancer (Hayashita et al. cancer Res 2005; Chow T F, J Urol. 2010; He et al Nature 2005), neuro- and medulloblastoma (Fontana et al. Plosone 2008; Uziel et al. PNAS 2009), pleural mesothelioma (Balatti V et al J Thorac Oncol. 2011). For its chromosomal location mir-17-92 could be very important in the pathogenesis of MM and has been described to confer tumorigenicity in MM (Chen L et al, Cancer Lett 2011). In fact miR-15a, miR16-1 and miR-17-92 cluster, located on 13q, play important roles in the regulation of cell proliferation, differentiation and apoptosis (Xiao Gao, et al. Leukemia Research, 2012). Moreover, it has been described that miR17-92 cluster is associated with the 13q gain (i.e. the amplification of the long arm (q) of chromosome 13) and c-myc expression during colorectal adenoma to adenocarcinoma progression (Diosdado B et al, Br J Cancer 2009).

Based on the sequence homology and seed conservation, the six miR-17-92 components belong to four distinct miRNA families, miR-17 (including miR-17 and 20), miR-18, miR-19 (including miR-19a and 19b), and miR-92 family (Olive V et.al. Immunological Reviews 2013). The distinct mature miRNA sequence of each miR-17-92 component determines the specificity of target regulation and ultimately the specificity of functional readout.

It is conceivable that the distinct biological effects of all four miRNA families will collectively contribute to the oncogenic activity of the miR-17-92 polycistron.

This unique gene structure underlies the unique functionality of miR-17-92 in a cell type and context-dependent manner. The individual miR-17-92 components perform distinct biological functions, which collectively regulate multiple related cellular processes during development and disease.

The structural complexity of miR-17-92 as a polycistronic miRNA oncogene, along with the complex mode of interactions among its components, constitutes the molecular basis for its unique functional complexity during normal and tumor development.

The functional significance of this gene organization is still largely unclear, yet it may implicate the existence of conserved functional interactions among specific miRNA components, giving rise to unusual gene regulatory mechanisms during cancer development (Olive V, et al. 2009. Genes & Dev; Gijs van Haaften and Reuven Agami. Genes Dev. 2010).

It is known that exists a functional interaction between c-Myc/n-Myc and the miR-17-92 cluster; both c-Myc and n-Myc can directly bind to the promoter of miR-17-92 and initiate transcription (O'Donnell K A, et al. Nature 2005). In the same way also all E2Fs, especially E2F3, have been shown to occupy miR-17-92's promoter region. This is a very important aspect because among the Transcription Factors, the E2F family (E2F1, E2F2 and E2F3) have a central role in the regulation of G1 to S phase progression, and E2Fs are also known to be targeted by miR-17-92, forming an auto-regulatory loop (E. Mogilyansky and I. Rigoutsos, Cell Death and Differentiation (2013).

The identification of miR-17-92 direct targets has been recently performed in lung and breast cancer cells by proteomic analysis approach (Kanzaki H et al Proteomics 2011; Ouchida M et al PlosOne 2012). Recent studies indicate that tumor protein p53 (TP53) targets the miR-17-92 cluster while also being targeted by miR-25 through regulation of the latter by Myc and E2F1 (Yan H L, et al.. EMBO J 2009). Phosphatase and tensin homolog (PTEN) and E2Fs were among the first validated miR-17-92 targets. The ability of the cluster's members to cooperate is evident in the context of TGF-b signaling. In particular, miR-17 and miR-20a directly target the TGF-b receptor II (TGFBRII), whereas miR-18a targets SMAD2 and SMAD4, two members of the TGF-b signaling pathway. TGF-b activation exerts an effect mediated in part by the cyclin-dependent kinase inhibitor (p21) and the apoptosis facilitator BCL2L11 (BIM), both of which are targeted by miR-17-92. In addition, BCL2L11 is targeted by miR-20a, miR-92, miR-19a and miR-19b (O′Donnell KA, Nature 2005; Ventura A, et al. Cell 2008), while miR-18a and miR-19 directly repress the antiangiogenic factors thrombospondin-1 (TSP-1) and connective tissue growth factor (CTGF). Moreover, miR-17 and miR-20a participate in the regulation of the insulin gene enhancer protein (151-1) and the T-box 1 protein (E Mogilyansky and I Rigoutsos, Cell Death and Differentiation 2013).

The specific processing of individual miRNAs adds a new level of complexity; it is conceivable that there is a cell-type dependent and context-dependent dimension to post-transcriptional silencing.

MiRNA expression patterns have relevance to the biological and clinical behavior of solid and hematologic tumors including multiple myeloma. Different reports suggest the role of this wide miRNAs family in human cancer. A number of three different miRs expression profiling dataset described miRNAs that belong to these clusters as up-regulated in MM (Pichiorri et al. PNAS 2008; Unno et al. Leuk Lymphoma 2009; Zhou et al PNAS 2010.) in lung, breast, stomach, prostate, colon and pancreatic cancers (Volinia S et al. PNAS 2006) or leukemia disease (Volinia S et al. Genome Res 2010)

Several evidences linked high levels of these miRNAs to poor prognosis in MM (Gao et al. Leuk Res 2012; Chen et al. Cancer Lett 2011, Wu et al. Br J Haematol 2013), in colon cancer (Yu G et al, J SUR Oncol 2010), in gastrointestinal cancer (Valladares-Ayerbes M et al, Int J Oncol 2011) The upregulation of miR-17-5p was associated with poor prognosis in pancreatic cancer (Yu J, et al. Cancer Biol Ther, 2010). In these cancer patients circulating miR-18a values are higher respect to controls and become lower in postoperative samples (Morimura R, Br J cancer 2011). Circulating mir-18a also contributes to cancer monitoring in gastric cancer patients (Tsujiura M, et al Gastric Cancer 2014). Finally, different miRNAs of the miR-17-92 cluster have been described as prognostic indicators and diagnostic tools in many cancers (Fassina A, Lab Invest 2012; Xu K L et al, Ann Thorac Surg 2014; Ohyashiki K et al plosOne 2011).

Therefore, the inhibition/suppression of the miRNAs belonging to the mir-17-92 cluster may be useful for the treatment of some tumors.

Some attempts to inhibit single miRNAs belonging to the miR-17-92 cluster have been made in the art.

The first evidence on the use of antagomir (antisense nucleotides) against miRNA members of miR-17-92 cluster has been reported in 2007. The authors demonstrated the induction of apoptosis in lung cancer cells by the use of an antisense oligonucleotide against miR-17-5p and miR-20a (Matsubara H Oncogene. 2007). So far, many reports described the inhibitions of cell proliferation and tumor progression in different human cancer overexpressing miR-17-92 by the use of inhibitors of single miR-17-92 family members such as miR-19a/b, miR-20a, miR-18a or miR-92a (Sharifi M, Mol Biol Rep. 2014; Murphy BL, Cancer Res. 2013; Wu Q, Cell Death Dis. 2014; Kang H W, FEBS Lett. 2012; Huang G, Cancer Res. 2012; Tao J, Mol Med Rep. 2012; Tsuchida A, Cancer Sc-i. 2011). Moreover, Pichiorri and coauthors demonstrated that the use of miR-19a/b inhibitors induced anti-MM activity in vitro and in vivo (Pichiorri et al. pnas 2008).

Molitoris et al (“Glucocorticoid-mediated repression of the oncogenic microRNA cluster miR-17-92 contributes to the induction of Bim and initiation of apoptosis”, MOLECULAR ENDOCRINOLOGY, vol. 25, no.3, 2011, pages 409-420) discloses 2′-O-methyl oligoribonucleotides with interspersed locked nucleic acids, targeting the individual miRNAs of the cluster.

Murphy et al (“Silencing of the miR-17-92 cluster family inhibits medulloblastoma progression”, CANCER RESEARCH, vol. 73, no. 23, 1 December 2013, pages 7068-7078) has shown that silencing mature miRNAs of the miR-17-92 cluster family by means of short LNA antagomirs inhibits medulloblastoma progression. In this work authors used anti-miR-17 and anti-miR-19 to inhibit mature miRNAs of the miR-17-92 cluster family.

E. Rao et al. (“The miRNA-17- 92 cluster mediates chemoresistance and enhances tumor growth in mantle cell lymphoma via PI3K/AKT pathway activation”, LEUKEMIA, 1 May 2012, pages 1064-1072) discloses that knockdown of miR-17 ˜92 expression, by the use of a doxycycline-inducible sponge lentiviral construct cell transduction, suppressed the PI3K/AKT pathway and inhibited tumor growth in a xenograft MCL mouse model.

These evidences show that the inhibition of one or more miRNAs belonging to the miR-17-92 cluster could be useful in tumor suppression.

The inhibition of miRNA transcriptional regulation however requires the overcoming of several challenges, which have not been solved yet.

In particular, an efficient miRNA inhibitor should have high binding affinity for the target miRNA, high nuclease resistance and be efficient for in vivo delivery.

In view of the above, an efficient tool for inhibiting the expression of the miRNAs of the miR-17-92 cluster in order to achieve an anti-tumoral activity is strongly desired.

So far, only the direct inhibition of individual mature miRNAs belonging to the miR-17-92 cluster has been disclosed in the art.

For example, WO2008014008 discloses antisense oligonucleotides targeting individual members (mature miRNAs) of the miR-17-92 cluster and their use for the therapy of certain types of cancers.

WO2006119365 discloses antisense miR-17-92 inhibitors for therapy of cancer. In particular, it is disclosed the activity of antisense oligonucleotides in B-cell malignancy or lymphoma wherein an high expression of miR-19-b-1 and miR-18 was determined.

WO2011106104 shows the anti-leukemic activity of antagomirs inhibiting individual mature miRNAs miR19a/b and miR-92.

WO2007095387 discloses RNA inhibitors tested for the ability to target six distinct mature miRNAs.

A tool which is able to simultaneously and efficiently inhibit more than one miRNA member belonging to the miR-17-92 cluster is still desired.

In particular, a tool which is able to simultaneously and efficiently inhibit all miRNA members belonging to the miR-17-92 cluster is even more desired.

The inventors of the present invention have found that the simultaneous inhibition of more than one miRNA, in particular of all miRNAs, of the miR-17-92 cluster can be achieved by inhibiting the primary transcript (pri-miRNA) of the miR-17-92 cluster so that it can not lead to the mature miRNAs.

However, the inhibition of such a pri-miRNA is further complicated by its length. Different chemically modified oligonucleotides including 2′-O-methyl, locked nucleic acid (LNA) or 3′ cholesterol-conjugated with full o partial phosphorothioate backbone have been proposed in the art to silence miRs in vivo. In addition, tiny LNAs and fully LNA-modified phosphorothioate oligonucleotides, designed with perfect complementary miRNAs seed sequence, have been disclosed and used as miRNAs knockdown in different tumor model in vivo.

LNA oligonucleotides are synthesized in different formats, such as all-LNA, LNA/DNA mixmers, or LNA/DNA gapmers. The first two types act by introducing a steric block to interfere with the transcriptional or translational machineries. The latter acts by attracting RNase H to the RNA/DNA heteroduplex for specific degradation of the target RNA by this enzyme (Grunweller et al, Biodrugs 2007).

The inventors of the present invention have now found that LNA/DNA gapmers specific for a region of the miR-17-92 pri-miRNA are able to exert an anti-tumoral activity through inhibition of the maturation of the miR-17-92 pri-miRNA and consequent downregulation of all the cluster members.

In particular, it has been found that targeting the miR-17-92 pri-miRNA is more efficient and advantageous in terms of growth and survival of tumoral cells with respect to targeting the single mature miRNAs.

### SUMMARY OF THE INVENTION

An inhibitor of the primary miRNA (pri-miRNA) of the miR-17-92 cluster for use in the treatment of tumors related to an overexpression of any of the miRNAs of the miR-17-92 cluster is an object of the present invention.

A preferred inhibitor of said pri-miRNA is a LNA/DNA gapmer which is able to specifically bind to a region of said pri-miRNA.

Preferably, said LNA/DNA gapmer is selected from the group consisting of the nucleotide sequences of the following Table 1, wherein letters with symbol “+” indicate the positions of LNA and symbol “*” indicates phosphorothioate bonds:

TABLE 1

| SEQ. | Name | |
|---|---|---|
| ID | (length) | Sequence (5′→3′) |
| 1 | miR-17-92 | +C*+T*+G*T*A*A*G*C*A*C* |
| | Gapmer_01 | T*T*T*+G*+A*+C |
| | (16 mer) | |
| 2 | miR-17-92 | +A*+C*+A*T*C*G*A*C*A*C* |
| | Gapmer_02 | A*A*+T*+A*+A |
| | (15 mer) | |
| 3 | miR-17-92 | +T*+C*+A*G*T*A*A*C*A*G* |
| | Gapmer_05 | G*A*C*+A*+G*+T |
| | (16 mer) | |
| 4 | miR-17-92 | +T*+A*+C*T*T*G*C*T*T*G* |
| | Gapmer_06 | G*+C*+T*+T |
| | (14 mer) | |
| 5 | miR-17-92 | +A*+T*+G*C*A*A*A*A*C*T* |
| | Gapmer_10 | A*A*C*+A*+G*+A |
| | (16 mer) | |
| 6 | miR-17-92 | +G*+A*+A*G*G*A*A*A*T*A* |
| | Gapmer_12 | G*C*A*+G*+G*+C |
| | (16 mer) | |
| 7 | miR-17-92 | +A*+G*+C*A*C*T*C*A*A*C* |
| | Gapmer_15 | A*T*C*+A*+G*+C |
| | (16 mer) | |
| 8 | miR-17-92 | +C*+G*+A*C*A*G*G*C*C*G* |
| | Gapmer_16 | A*A*+G*+C*+T |
| | (15 mer) | |

A more preferred inhibitor is a LNA/DNA gapmer having the following nucleotide sequence: +T*+A*+C*T*T*G*C*T*T*G*G*+C*+T*+T* [SEQ ID N. 4].

The LNA/DNA gapmers listed in Table 1 are also objects of the present invention.

It has been found that the use of a molecule targeting the primary miRNA (pri-miRNA) of the miR-17-92 cluster is extremely advantageous with respect to the targeting of single miRNAs. First of all, because a single molecule can be used to inhibit more than one miRNA of the cluster. More importantly, it has been found that a molecule targeting the pri-miRNA has a more effective anti-tumoral activity with respect to molecules targeting single miRNAs of the cluster.

More in particular, it has been found that the inhibition of the pri-miRNA can be efficiently achieved by using a LNA/DNA gapmer which is able to selectively bind to the pri-miRNA and at the same time to recruit the RNase H, which specifically degrades the targeted pri-miRNA.

Even more in particular, the inventors of the present invention have found some LNA/DNA gapmers which are able to specifically bind to a region of the miR-17-92 pri-miRNA and induce its degradation, thus leading to inhibition of one or more miRNA members of the miR-17-92 cluster (miR-17, miR-18a, miR-19a/b, miR-20a and miR-92a).

Thanks to the presence of both the DNAs and the LNAs these LNA/DNA gapmers have the advantages of an enhanced selectivity and efficiency in their inhibitory activity on the target pri-miRNA and at the same time show a remarkable improvement in stability, thus allowing their systemic delivery.

This last advantage is of major relevance since it makes them suitable for clinical application, where intravenous administration is the conventional route.

The LNA/DNA gapmers of the invention have shown an anti-tumoral activity thanks to their ability to inhibit the miR-17-92 pri-miRNA.

They can thus be used for the treatment of tumors related to an overexpression of any of the miRNAs belonging to the miR-17-92 cluster.

In particular, it is an object of the present invention an inhibitor of the miR-17-92 cluster for use in the treatment of a tumor selected from the group consisting of: multiple myeloma, Waldenstrom Macroglobulinemia, mesothelioma, bladder cancer, B-cell Lymphomas, B-cell Chronic Lymphocytic Leukemia, Acute Myeloid Leukemia, T-cell Lymphoma, Retinoblastoma, Osteosarcoma, Colorectal Cancer, Head and Neck Cancers, Pancreatic Cancer, Breast Cancer, Ovarian Cancer, Lung Cancer, Renal Cancer and Hepatocellular Carcinoma.

In a preferred embodiment, said tumor is selected from the group consisting of multiple myeloma, Waldenstrom Macroglobulinemia, pancreatic cancer, breast cancer, lung cancer, bladder cancer and pleural mesothelioma cancer.

As mentioned above, the use of the LNA/DNA gapmers for the treatment of the above mentioned diseases of the invention provides the advantages of a successful delivery and a powerful anti-tumor activity.

Means of delivery of said LNA/DNA gapmers are also within the scope of the present invention.

### DESCRIPTION OF THE INVENTION

Definitions

Within the context of the present invention, the term “microRNA” or “miRNA” or “miR” means a short ribonucleic acid (RNA) molecule found in eukaryotic cells.

Within the context of the present invention, the terms “miRNA inhibitors” refer to molecules which bind and inhibit one or more specific mature miRNAs.

Within the context of the present invention, the terms “primary transcript”, “pri-miRNA” and “primary miRNA” are synonymous and they all designate the single-stranded ribonucleic acid (RNA) product synthesized by transcription of DNA, and processed to yield various mature RNA products such as miRNAs, tRNAs, and rRNAs. In particular, the primary transcript of the miR-17-92 cluster is the single-stranded ribonucleic acid (RNA) product synthesized by transcription of the miR-17- 92 cluster gene.

Within the context of the present invention, the term “Locked Nucleic Acid” or “LNA” refers to a nucleotide with the ribose ring locked in an N-type conformation by a 2′-O, 4′-C methylene bridge. LNA units are described in inter alia WO 99/14226, WO 00/56746, WO 00/56748, WO 01/25248, WO 02/28875, WO 03/006475, WO 03/095467 and references cited therein.

Within the context of the present invention, a PS-oligonucleotide is an oligonucleotide with a phosphorothioate bond, where a phosphorothioate bond is the substitution with a sulfur atom of a non-bridging oxygen in the phosphate backbone of said oligonucleotide.

Within the context of the present invention, the term “gapmers” or “LNA/DNA gapmers” refers to fully PS-oligonucleotides with a central DNA moiety flanked by LNA modified 5′- and 3′- ends.

Within the context of the present invention, multiple myeloma is a cancer of plasma cells characterized by abnormal proliferation of neoplastic plasma cells in the bone marrow.

Within the context of the present invention, a tumor related to an overexpression of any of the miRNAs of the miR-17-92 cluster is a tumor wherein an overexpression of at least one miRNA belonging to this cluster has been or can be detected by techniques known in the art, for example by widely accepted assays as PCR-based technique (Schmittgen T D, NAR, 2004), bead-based flow-cytometry (Lu J., Nature, 2005) and microarray (Liu et al, 2004).

For example, a tumor related to an overexpression of any of the miRNAs of the miR-17-92 cluster can be selected from the group consisting of: multiple myeloma, Waldestrom Macroglobulinemia, mesothelioma, bladder cancer, B-cell Lymphomas, B-cell Chronic Lymphocytic Leukemia, Acute Myeloid Leukemia, T-cell Lymphoma, Retinoblastoma, Osteosarcoma, Colorectal Cancer, Head and Neck Cancers, Pancreatic Cancer, Breast Cancer, Ovarian Cancer, Lung Cancer, Renal Cancer and Hepatocellular Carcinoma (E Mogilyansky and I Rigoutsos, Cell Death and Differentiation, 2013).

### FIGURES

FIG. 1. Relative expression of miR-17-92 cluster in MM cell lines. q-RT-PCR of miR-17-92 member family and of two pri-miRs, specifically designed for quantification of the primary transcript of the miR-17-92 cluster, assessed in a panel (11) of MM cell lines. The results are shown as average miRNA expression after normalization with RNU44 and ΔCt calculations. Data represent the average of 3 independent experiments ±SD.

FIG. 2. In vitro screening of eight miR-17-92 LNA Gapmers. A) q-RT-PCR of miR-17-92 member family and of two pri-miRs after 48 hours of transfection with 25 nM of the eight gapmer oligonucleotides listed in Table 1 and RNase-free water as control, in NCI-H929 MM cells. The results are shown as average miRNA expression after normalization with RNU44 and ΔΔCt calculations and are reported as miRNAs expression level relative to RNase-free water. Data represent the average of 3 independent experiments ±SD. B) q-RT-PCR of miR-17-92 member family and of two pri-miRs, after 48 hours of transfection with 25 nM of the two gapmers (miR-17-92 LNA gapmer_06 and gapmer_15) that shown in A the strongest downregulation of primary transcripts and miR-17-92 mature members, in RPMI-8226 MM cells. The results are shown as average miRNA expression after normalization with RNU44 and ΔΔCt calculations and are reported miRNAs expression level relative to RNase-free water. Data represent the average of 3 independent experiments ±SD. C-D) Effects induced on proliferation (C, as assessed by CCK-8 assay) and apoptosis (D, as assessed by AnnexinV/7-AAD staining) in RPMI-8226, KMS-12-BM and NCI-H929 MM cells, induced after 48 hours of transfection with 25 nM of the eight LNA gapmer oligonucleotides. The strongest biological effect was induced by the oligonucleotide named gapmer_06 (white bar and black line) in all MM cell lines analyzed. Averaged values ±SD from 3 independent experiments are represented.

FIG. 3. Gapmer_06 is a specific inhibitor of the miR-17-92 cluster of microRNAs. A) q-RT-PCR of miR-17-92 member family and of the two pri-miRs, 48 hours after transfection with either 25 nM of gapmer_06 or scrambled gapmers or mismatched gapmers or RNase-free water as control, in NCI-H929 MM cells. Only the transfection with miR-17-92 LNA gapmer_06 (white bar) induce downregulation of the two primary transcript and mature single miRNA members as compared to control. The results are shown as average miRNA expression after normalization with RNU44 and ΔΔCt calculations and are reported as miRNAs expression level relative to RNase-free water. Data represent the average of 3 independent experiments ±SD. B) Flow cytometry analysis of 7-AAD stained NCI-H929 cells shows that transfection with both scrambled LNA gapmers and mismatched LNA gapmers does not induce cell death while transfection with gapmer_06 reduces the live cells by 20%, as compared to RNase-free water. C) q-RT-PCR of miR-17-92 member family and of two pri-miRs, 48 hours after transfection with either 25 nM of gapmer_06 or non phosphorothioated (NP) gapmer_06 (25 and 100 nM) or RNase-free water, in NCI-H929 MM cells. Transfection with NP gapmer_06 requires higher concentrations (100 nM) to induce a down-regulation of the two primary transcripts and mature single miRNA members, as compared to gapmer_06 (25 nM), The results are shown as average miRNA expression after normalization with RNU44 and ΔΔCt calculations. Data represent the average of 3 independent experiments ±SD. D) Flow cytometry analysis of 7-AAD NCI-H929 stained cells confirms that transfection with NP gapmer requires higher concentrations (100 nM) to induce cell death, as compared to gapmer_06 (25 nM). E) q-RT-PCR of miR-17-92 member family and of two pri-miRs, 48 hours after transfection with either 25 nM of gapmer_06 or mixmer_06 or RNase-free water, in NCI-H929 MM cells. Only the transfection with miR-17-92 gapmer_06 (white bar) induces downregulation of the two primary transcript and mature single miRNA members as compared to the mixmer_06. The results are shown as average miRNA expression after normalization with RNU44 and ΔΔCt calculations. Data represent the average of 3 independent experiments ±SD. F) Flow cytometry analysis of 7-AAD NCI-H929 stained cells shows that LNA mixmer_06 does not induce cell death.

FIG. 4. Antiproliferative activity of LNA gapmer_06 on different MM cell lines. Cell growth analysis of 12 MM cell lines transfected with 3 different concentrations of LNA gapmer (50-25-12,5 nM). Analysis was performed by CCK-8 assay. Averaged values of three independent experiments are plotted including ±SD.

FIG. 5. Different strategies in comparison: targeting the pri-miR-17-92 by LNA gapmers versus targeting the single miR-17-92 members by different antisense oligonucleotides. A) Cell growth analysis of RPMI-8226, KMS-12-BM and NCI-H929 cells transfected with 25 nM of either gapmer_06 or single miRNA inhibitors for miR-17/18a/19a/19b/20a/92a. Analysis was performed by CCK-8 assay. Averaged values of three independent experiments are plotted including ±SD. B) Cell viability analysis of RPMI-8226, KMS-12-BM and NCI-H929 cells transfected with 25 nM of either LNA gapmer_06 or miRNA inhibitors for miR-17/18a/19a/19b/20a/92a. Analysis was performed by flow cytometry in 7-AAD stained cells. Averaged values of three independent experiments are plotted including ±SD.

FIG. 6. Transfectant reagents-free (gymnotic) delivery of gapmers to MM cells. A) q-RT-PCR of miR-17-92 family members and of the two pri-miRs, in NCI-H929 cells after 6 days of continuous transfectant reagents-free (gymnotic) exposure to 1 and 2,5 μM of gapmer_06. Gymnotic exposure to gapmers induced the downregulation of the two primary transcripts and mature single miRNA family members in a dose dependent manner in both cell lines. The results are shown as average miRNA expression after normalization with RNU44 and ΔΔCt calculations and are reported as miRNAs expression level relative to RNase-free water. Data represent the average of 3 independent experiments ±SD. B) Cell growth analysis of 11 MM cell lines gymnotically exposed for 6 days to LNA gapmer_06 at concentrations ranging from 0 to 20 μM. Analysis was performed by CCK-8 assay. C) Cell viability analysis of 11 MM cell lines gymnotically exposed for 6 days to LNA gapmer_06 at concentrations ranging from 0 to 20 μM. Analysis was performed by flow cytometry in 7-AAD stained cells. Averaged values of three independent experiments are plotted including ±SD.

FIG. 7. LNA gapmer is active against CD138+primary MM cells. A) Cell viability analysis of CD138+primary cells, derived from bone marrow aspirates of 14 myeloma patients, gymnotically exposed for 6 days to 0-2,5-5-10 μM of gapmer_06; B) Cell viability analysis of CD138+primary cells, derived from bone marrow aspirates of 3 MGUS patients, gymnotically exposed for 6 days to 5 μM of gapmer_06; C) Cell viability analysis of PBMCs, derived from 2 healthy donors, gymnotically exposed for 6 days to 5 μM of LNA gapmer_06. Analysis was performed by flow cytometry in 7-AAD stained cells.

FIG. 8. LNA gapmer synergizes with proteosome inhibitors and melphalan to induce cell death in AMO1 cell line. A) AMO1 cells were exposed for 6 days to LNA gapmers and/or Bortezomib and then cell viability was evaluated by flow cytometric analysis of 7-AAD staining. B) AMO1 cells were exposed for 6 days to LNA gapmers and/or Carfilzomib and then cell viability was evaluated by flow cytometric analysis of 7-AAD staining. C) AMO1 cells were exposed for 6 days to LNA gapmers and/or Melphalan and then cell viability was evaluated by flow cytometric analysis of 7-AAD staining .The percentage of living cell is shown as compared to the untreated cells. Averaged values of three independent experiments are plotted including ±SD.

FIG. 9. Molecular perturbations affected by miR-17-92 LNA gapmer_06 in myeloma cells. NCI-H929 cells were exposed for 6 days to gapmers or scrambled controls, at a concentration of 2,5 μM. A) Western Blot analysis of PTEN, AKT and ERK proteins and their phosphorilated/active isoforms (p-AKT and p-ERK). B) Western Blot analysis of E2F-1 and p14/ARF-p53 “fail-safe”; C) Western Blot analysis of intrinsic apoptotic pathway including pro-apoptotic proteins (Puma, Bak, Bax, Bid and Bim) and anti-apoptotic proteins (Bcl-2, Mcl-1). Caspase-3 cleavage is also showed. Each experiment was performed at least 3 times. A representative one is shown. GAPDH was used as protein loading control.

FIG. 10. In vivo anti-tumor activity of gapmer_06 in MM xenografted SCID/NOD mice. A) In vivo anti-MM activity of gapmer_06 was evaluated in NCI-H929 xenografted SCID.NOD mice. Mice were randomized in 6 different groups, to receive intraperitoneally: saline, gapmer_06 at concentration of 75-50-25 μg once weekly or 25-12,5 μg twice weekly. Tumors were measured with an electronic caliper every 3 days, averaged tumor volumes of each group ±SD are shown. B) Survival curves (Kaplan-Meier) show a survival advantage in mice treated with gapmer_06 as compared to controls (log-rank test, P<0.05). Survival was evaluated from the first day of treatment until death or sacrifice. Percent of mice alive is shown. C). In vivo tumor growth of AMO1 xenografts I.P treated with 2 mg/kg LNA gapmer_06, or Bortezomib 1 mg/kg or NaCl as control. I.P injection were administrated at day 1-4-8-15-22. Tumors were measured using IVIS LUMINA II Imaging System after 11-18-25 days from the beginning of treatment. The pictures show the in vivo detection of the tumor volume in representative AMO1 xenografts of saline solution NaCl 0,9% (control) group (5 mice), gapmer_06 2 mg/kg treatment group (5 mice) and Bortezomib 1 mg/kg treatment group (3 mice) using IVIS LUMINA II Imaging System. Pictures show measurement of tumor volumes made at the beginning of treatments (T0) and 11 days (T1) from treatments. Average tumor volume of each group ±SD is shown in the graph. P values were obtained using two-tailed t test (* indicates p<0.05). D) In vivo tumor growth of AMO1/abzb xenografts I.P treated with gapmer_06 2 mg/kg, or Bortezomib 1 mg/kg, or NaCl as control. I.P injection were administrated at day 1-4-8-15-22. The pictures show the in vivo detection of the tumor volume in AMO¹/abzb xenografts of saline solution (control) group (4 mice), LNA gapmer_06 2 mg/kg treatment group (5 mice) and Bortezomib 1 mg/kg treatment group (4 mice) using IVIS LUMINA II Imaging System. Pictures show measurement of tumor volumes made at the beginning of treatments (T0) and 11 days (T1) from treatments. Average tumor volume of each group ±SD is shown. P values were obtained using two-tailed t test (* indicates p<0.05). E) In vivo tumor growth of U266 orthotopic model I.P treated with LNA gapmer_06 2 mg/kg or NaCl as control. I.P injection were administrated at day 1-4-8-15-22. Tumors were measured using IVIS LUMINA II Imaging System after 4-6-10 weeks after cell injection. The pictures show the in vivo detection of the tumor volume in U266 orthotopic model of saline solution (control) group (5 mice) and G06 2 mg/kg treatment group (4 mice) using IVIS LUMINA II Imaging System. Measurement of tumor volumes were made 15 days after intravenously cells injection (T0), at the beginning of treatments, one week later first measurement (T1), and 11 days from treatments (T2). Average tumor volume of each group ±SD is shown. P values were obtained using two-tailed t test (* indicates p<0.05). F) SCID-hu mice engrafted with INA-6 cells were monitored for tumor growth by serial serum measurements of shulL-6R at days 0-20-40-60 from cell injection. Antitumor effects were determined after treatment at day 1-4-8-15-22 with LNA gapmer_06 2 mg/kg. Mice were divided into a control group (3 mice) and a gapmer_06 2 mg/kg I.P treated group (3 mice). Average soluble IL6-R concentration of each group ±SD is shown. P values were obtained using two-tailed t test (* indicates p<0.05).

FIG. 11. Toxicity in vivo evaluation. BALB/c mouse (Harlan Laboratories) were treated with 2 mg/kg at days 1, 4, 7, 14 and 22 after palpable tumors were detected. Hematoxylin and eosin staining (40-fold magnification) of kidney, liver and bone marrow, retrieved from mice treated with miR-17-92 LNA gapmer_06 or saline are shown.

FIG. 12. Gymnotic exposure to LNA gapmers induce antiproliferative activity on different cancer cell lines in vitro. Effects induced on proliferation, as assessed by CCK-8 assay, after 6 days of gymnotic exposure to 2.5, 5 or 10 μM of LNA gapmers on Capan1 pancreatic cancer cells, MCF7 and MDA231 breast cancer cells, LXF-289, A-549 and NCI-H82 lung cancer cells, 5637 bladder cancer cells, BCMW.1 Waldenstrom Macroglobulinemia cells and MPM209 mesothelioma cells. The percentage of living cells is shown as compared to the correspondent untreated cancer cells. Averaged values of three independent experiments are plotted including ±SD. The antiproliferative activity enrich significant value (P<0.05) in the different cell types tested and at all the different concentration used.

FIG. 13. A) The body weight of each monkey recorded on the day of the first administration and in weekly intervals thereafter, at the same day of the week. B) Quantity of food left by individual animals, recorded on a daily basis throughout the experimental period. From these data the quantities consumed were calculated. The graph represents the food consumption recorded on the day of the first administration and in weekly intervals thereafter, at the same day of the week. C) Hematology parameters were determined in the test week -1 (baseline) and before dosing on test days 4, 8, 15, 22. The determinations were performed by ADVIATM 120 (Siemens Diagnostics GmbH, Fernwald, Germany). The graphs report some of the parameters evaluated including the red and white blood cells, the hemoglobin content and the platlets. D) Clinical chemistry evaluations were performed by KONELAB 30i (Thermo Fisher Scientific, Dreieich, Germany) with the same timing described in (C). The graphs report some of the parameters evaluated including alanine and aspartate aminotransferase, bilirubine and creatinine.

### DETAILED DESCRIPTION OF THE INVENTION

The sequences of miR-17-92 cluster members are available in the state of the art with the IDs hsa-miR-17, hsa-miR-18a, hsa-miR-19a, hsa-miR-20, hsa-miR-19b and hsa-miR-92a (www.microrna.orq; August 2010 Release).

The sequence of the miR-17-92 primary transcript is known in the art and it is available in the NIH genetic sequence database Genbank with the accession number AB176708 (website: http://www.ncbi.nlm.nih.gov/nuccore/AB176708).

An inhibitor of the primary miRNA (pri-miRNA) of the miR-17-92 cluster for use in the treatment of tumors related to an overexpression of any of the miRNAs of the miR-17-92 cluster is an object of the present invention.

A preferred inhibitor of said pri-miRNA is a LNA/DNA gapmer which is able to specifically bind to a region of said pri-miRNA.

Preferably, said LNA/DNA gapmer is selected from the group consisting of the nucleotide sequences of Table 1, above reported, wherein letters with symbol “+” indicate the positions of LNA and symbol “*” indicates phosphorothioate bonds.

All the LNA/DNA gapmers of Table 1 have a fully phosphorothioted (PS)-modified backbone, with a DNA core flanked by LNA™.

The PS-oligonucleotides introduced at the 5′ end of the oligo inhibit exonuclease degradation while PS-oligonucleotides introduced internally limit attack by endonucleases.

The LNA part increases the affinity for the target and confers nuclease resistance, while the DNA core of the oligo activates the RNase H.

LNA/DNA gapmers thus bind to a specific region of the pri-miRNA, causing the recruitment of RNase H. As a consequence, the pri-miRNA is degraded and fails to generate mature miRNAs.

Modifications of the backbone of the inhibitors, of the present invention which do not substantially affect their activity are also within the scope of the invention. In particular, possible modifications can be in the position of the phosphorothioate (PS) bonds and of the LNA nucleotides as far as these modifications do not affect the activity of the LNA/DNA gapmer.

The LNA/DNA oligonucleotides of the invention exhibit thermal stability when hybridized to the complementary RNA strand. Indeed, the melting temperature (TM) increases 2-8° C. for each incorporated LNA-monomer.

The novel LNA/DNA gapmers of the present invention have the primary advantage of being able to inhibit the primary transcript (pri-miRNA) of the miR-17-92.

In particular, the specific structure of the LNA/DNA gapmers of the invention provides for an enhancement of selectivity and efficiency of the inhibitory activity on the target sequence and at the same time for a remarkable improvement of the stability of the inhibitor.

This high stability allows overcoming many potential pharmacokinetic and pharmacodynamic issues which may arise from the use of delivery systems.

This enhanced stability also allows for the systemic delivery of said inhibitors.

This last advantage of the novel inhibitors of the present invention is of major relevance since it makes them suitable for clinical application, where systemic administration is the conventional route.

LNA/DNA gapmers act as antisense oligonucleotides by recognizing a target sequence. Antisense oligonucleotides may have the disadvantage of retaining off-target effects, other than the antisense activity. This is especially true for the new generation compounds, such as LNA/DNA gapmers, due to the chemical modifications introduced to enhance their stability and target affinity.

It has been found that the balance between in-targets and off-target effects, which is the effectiveness/toxicity balance, is satisfactory for the LNA/DNA gapmers of the invention. This is of particular relevance for their clinical application and for their use in clinical studies.

Furthermore, the LNA/DNA gapmers of the invention are successfully uptaken by the cells also when transfection is in reagents-free conditions (gymnotic delivery). This has been confirmed by the downregulation of the pri-miR and all mature miRNAs that belong to miR-17-92 cluster in gymnotically transfected cells. Gymnotic delivery is a known technique of delivery which takes advantage of the normal growth properties of cells in tissue culture and which has been reported to correlate particularly well with in vivo targeting activity (Stein CA et al, NAS 2010).

Therefore, the inhibitors of the invention have the further advantage of being effective in vitro also when cells are transfected without any reagent.

Also, the efficient gymnotic delivery, as mentioned above, is a further indicator of the good in vivo targeting activity of the inhibitors of the invention.

The downregulation at protein level of canonical targets of miR-17-92 cluster members, after gymnotic exposure of Multiple myeloma (MM) cells to the LNA/DNA gapmers of the invention, was also achieved further supporting the anti-tumoral activity of said inhibitors.

The LNA/DNA gapmers of the invention are able to inhibit tumor growth both in vitro and in vivo in MM models.

When used in vivo the inhibitors of the present invention provide the advantage of exercising an effective anti-tumor activity in multiple myeloma when systemically administered.

The LNA/DNA gapmers of the invention are also able to inhibit tumor growth in breast, lung, pancreatic and bladder cancer cells as well as in pleural mesothelioma and Waldenstrom macroglobulinemia cells.

The present invention provides therefore said LNA/DNA gapmers for use for the treatment of a tumor selected from the group consisting of multiple myeloma, Waldenstrom Macroglobulinemia, pancreatic cancer, breast cancer, lung cancer, bladder cancer and pleural mesothelioma cancer.

The inhibitors of the invention can be administered as such or by means of a suitable vector known for the administration of RNA or DNA. An exemplary vector is the adeno-associated vector (AAV), a well-known viral vector for administration of DNA in vivo. Methods and formulations of this kind are conventional and well known in the art and do not need any further explanation.

The inhibitors of the invention can be administered as a medicament, for example in a pharmaceutical composition.

A pharmaceutical composition comprising the LNA/DNA gapmers of the invention is therefore also an object of the present invention. The composition contains as active ingredient at least one inhibitor according to the present invention and a suitable carrier. Average quantities of the active ingredient may vary and in particular should be based upon the recommendations and prescription of a qualified physician.

It is also an object of the present invention, a pharmaceutical composition comprising one or more inhibitors of the primary miRNA of the miR-17-92 cluster as active ingredients for the treatment of tumors related to an overexpression of any of the miRNAs of the miR-17-92 cluster.

In particular, said pharmaceutical composition can be for the treatment of a tumor selected from the group consisting of: multiple myeloma, Waldenstrom Macroglobulinemia, mesothelioma, bladder cancer, B-cell Lymphomas, B-cell Chronic Lymphocytic Leukemia, Acute Myeloid Leukemia, T-cell Lymphoma, Retinoblastoma, Osteosarcoma, Colorectal Cancer, Head and Neck Cancers, Pancreatic Cancer, Breast Cancer, Ovarian Cancer, Lung Cancer, Renal Cancer and Hepatocellular Carcinoma.

In a preferred embodiment, said pharmaceutical composition is for the treatment of a tumor selected from the group consisting of multiple myeloma, Waldenstrom Macroglobulinemia, pancreatic cancer, breast cancer, lung cancer, bladder cancer and pleural mesothelioma cancer.

A pharmaceutical composition comprising at least one of the LNA/DNA gapmers listed in table 1 is also an object of the present invention.

The inventors of the present invention have also found that when inhibitors of the miR-17-92 cluster are administered together with proteasome inhibitors a synergistic effect, in particular in the treatment of multiple myeloma, is observed.

Proteasome inhibitors are known in the treatment of multiple myeloma. Examples of suitable proteasome inhibitors are bortezomib and carfilzomib.

Therefore, it is also an object of the present invention, a pharmaceutical composition comprising the LNA/DNA gapmers listed in table 1 and one or more proteasome inhibitors compound.

Preferably, such proteasome inhibitor is selected from bortezomib and carfilzomib.

In a preferred embodiment, said pharmaceutical composition is for use for the treatment of multiple myeloma.

A further object of the invention is a pharmaceutical composition comprising an inhibitor of the pri-miRNA of the miR-17-92 cluster and one or more proteasome inhibitors compound for the treatment of multiple myeloma.

It has further been found that the inhibitors of the present invention have an anti-myeloma activity also on tumors refractory to the treatment with proteasome inhibitors.

This provides a further advantage to the use of such inhibitors since they can be effectively used also in the treatment of tumors not responding to other therapies.

The pharmaceutical composition according to the present invention can also comprise further active ingredients.

In particular, the inventors of the present invention have found that when inhibitors of the miR-17-92 cluster are administered together with active ingredients with an anti-myeloma activity a synergistic effect in the treatment of multiple myeloma is observed.

Therefore, it is also an object of the present invention a pharmaceutical composition comprising an inhibitor of the pri-miRNA of the miR-17-92 cluster and an active ingredient with anti-myeloma activity for the treatment of multiple myeloma.

Exemplary active agents with anti-myeloma activity are: melphalan, prednisone, thalidomide, cyclophosphamide, bendamustine, doxorubicin, lenalidomide, pomalidomide.

A preferred anti-myeloma active ingredient is melphalan (IUPAC name: 4-[bis(chloroethyl)amino]phenylalanine).

In a preferred embodiment, said pharmaceutical composition comprises the LNA/DNA gapmers listed in table 1 and an anti-myeloma active ingredient, preferably melphalan.

Even more preferred is a pharmaceutical composition comprising the gapmer with SEQ ID N.4 (gapmer_06, also named miR-17-92-i-PT) and melphalan.

The skilled in the art can decide the effective amount of anti-myeloma active agent or proteasome inhibitor to use in combination with the inhibitors of the present invention, according to its general knowledge in the field.

Exemplary amounts for the anti-myeloma active agent may vary from about 0.5 to about 2 μM, preferably from about 1.5 to about 2 μM.

For bortezomib exemplary amounts may vary from about 0.002 to about 0.0035 μM, preferably it can be about 0.0035 μM.

For carfilzomib exemplary amounts may vary from about 0.0004 to about 0.0007 μM.

In such combinations, the inhibitor of the invention, in particular the LNA/DNA gapmer of Table 1, can be administered, for example, in an amount comprised between about 0.5 and about 1.25 μM.

The pharmaceutical compositions according to the present invention contain, along with the active ingredient, at least one pharmaceutically acceptable vehicle or excipient.

A preferred vehicle is saline solution.

According to the present invention, the inhibitors can be administered as a medicament to a subject suffering from multiple myeloma or from any other tumor related to an overexpression of the miR-17-92 cluster by conventional methods.

Conveniently, said medicament is in the form of a preparation for systemic administration but other forms are equally suitable for carrying out the present invention. The person skilled in the art will decide the effective time of administration, depending on the patient's conditions, degree of severity of the disease, response of the patient and any other clinical parameter within the general knowledge of this matter. Reference can be made to Remington's Pharmaceutical Sciences Handbook, last edition.

In vivo experiments have been performed by intraperitoneal administration since it is convenient for the experimental setting. However, intraperitoneal administration is not much suitable for clinical practice, where an intravenous administration is instead preferred. Intraperitoneal route is considered almost equivalent to the intravenous route due to the almost total blood clearance of intraperitoneally administered biomolecules.

According to the administration route chosen, the compositions will be in solid or liquid form, suitable for oral, parenteral or intravenous administration.

The following examples further illustrate the invention.

### EXAMPLES

Materials and Methods

Cell Lines

Human cell lines were grown at 37° C., 5% CO2, in media with antibiotics.

Specifically:

Myeloma cells: NCI-H929, MM.1S, MM.1R, U266, U266/LR7, RPMI-8226, RPMI-8226/DOX40, INA-6, XG-1, OPM2,KMS-12-BM, AMO1, AMO1/abzb and AMO1/acfz were cultured in RPMI-1640 medium (GibcoH,Life Technologies, Carlsbad, Calif.,USA) supplemented with 10% fetal bovine serum, 100 U/ml penicillin, and 100 mg/ml streptomycin (GibcoH); IL-6 dependent cell lines INA-6 and XG-1 were cultured in the presence of rhIL-6 (R&D Systems, Minneapolis, Minn.), as previously reported.

Pancreatic cancer cells: Capan1 were cultured in RPMI-1640 medium (GibcoH,Life Technologies, Carlsbad, Calif.,USA) supplemented with 10% fetal bovine serum, 100 U/ml penicillin, and 100 mg/ml streptomycin (GibcoH).

Breast cancer cells: MCF7 were cultured in ATCC-formulated Eagle's Minimum Essential Medium, supplemented with 0.01 mg/ml human recombinant insulin, 10% fetal bovine serum, 100 U/ml penicillin, and 100 mg/ml streptomycin (GibcoH); MDA-231 were cultured in ATCC-formulated Leibovitz's L-15 Medium, supplemented with 10% fetal bovine serum, 100 U/ml penicillin, and 100 mg/ml streptomycin (GibcoH);

Lung cancer cells: A-549, LXF-289 (NSCLC) and NCI-H82 (SCLC) were cultured in RPMI-1640 medium (GibcoH,Life Technologies, Carlsbad, Calif.,USA) supplemented with 10% fetal bovine serum, 100 U/ml penicillin, and 100 mg/ml streptomycin (GibcoH);

Bladder cancer cells: 5637 were cultured in RPMI-1640 medium (GibcoH,Life Technologies, Carlsbad, Calif.,USA) supplemented with 10% fetal bovine serum, 100 U/ml penicillin, and 100 mg/ml streptomycin (GibcoH);

Waldenstrom Macroglobulinemia cells: BCMW1 were cultured in RPMI-1640 medium (GibcoH,Life Technologies, Carlsbad, Calif.,USA) supplemented with 10% fetal bovine serum, 100 U/ml penicillin, and 100 mg/ml streptomycin (GibcoH);

Pleural mesothelioma cells: MPM-209 were cultured in Dulbecco's Modified Eagle Medium (DMEM, GibcoH,Life Technologies, Carlsbad, Calif.,USA) supplemented with 10% fetal bovine serum, 100 U/ml penicillin, and 100 mg/ml streptomycin (GibcoH);

Human stromal cells: HS-5 were cultured in Dulbecco's Modified Eagle Medium (DMEM, GibcoH,Life Technologies, Carlsbad, Calif.,USA) supplemented with 10% fetal bovine serum, 100 U/ml penicillin, and 100 mg/ml streptomycin (GibcoH).

Primary Cells

Following informed consent approved by our University Hospital Ethical Commitee, CD138+cells were isolated from the BM aspirates of both Monoclonal gammopathy of undetermined significance (MGUS) and myeloma patients by Ficoll-Hypaque density gradient sedimentation followed by antibody-mediated positive selection using anti-CD138 magnetic activated cell separation microbeads (Miltenyi Biotech, Gladbach, Germany). Purity of immunoselected cells were assessed by flow-cytometric analysis using a phycoerythrin-conjugated CD138 monoclonal antibody by standard procedures. CD138+cells were seeded on HS-5 cells and cultured in RPMI-1640 medium (GibcoH,Life Technologies, Carlsbad, Calif.,USA) supplemented with 10% fetal bovine serum, 100 U/ml penicillin, and 100 mg/ml streptomycin (GibcoH);

Peripheral Blood Mononuclear Cells (PBMCs) from healthy donors were isolated from buffy coats by Ficoll-Hypaque density gradient sedimentation. PBMCs were cultured in RPMI-1640 medium (GibcoH, Life Technologies, Carlsbad, Calif.,USA) supplemented with 10% fetal bovine serum, 100 U/ml penicillin, and 100 mg/ml streptomycin (GibcoH).

In vitro Transfection of MM Cells

Commercial miRNA inhibitors for miR-17a, miR-18a, miR-19a, miR-19b, miR-20a and miR-92a were purchased from Ambion (Applied Biosystems, Calif., U.S.).

3×10⁵ cells were transfected using Neon Transfection System (Invitrogen, Calif.,U.S.), (2 pulses at 1150, 30ms). The transfection efficiency evaluated by flow-cytometric analysis relative to a FAM dye—labeled anti-miR—negative control reached 85% to 90%.

Gymnotic Delivery of LNA Gapmers

Cells were seeded at low plating density in order to reach confluence on the final day of the experiments (day 6). Cell number at plating ranged from 0.5 to 2.5×10³ in 96-well plates, from 2.5 to 10×10³ in 12-well plates and from 1 to 3×10⁴ in 6-well plates.

The LNA/DNA Gapmers were used at concentrations ranging from 0.1 to 20 μM.

Combination with Proteasome Inhibitors

Bortezomib (PS-341, catalog n° S1013) and Carfilzomib (PR-171, catalog n°S2853) were purchased from Selleck. Combination between miR-17-92 LNA gapmer_06 and proteasome inhibitors was tested in AMO1 cells. 1×10³ cells were plating in 96-well plates. Bortezomib was used at concentrations ranging from 2 nM to 3.5 nM. Carfilzomib was used at concentrations ranging from 0.4 to 0.7 nM. Measurement of synergistic index (SI) was determined with the following formula: SI=(effect induced by miR-17-92 LNA gapmers and proteasome inhibitors in combination)/effect of miR-17-92 LNA gapmers) +(effect of proteasome inhibitors). Interactions were considered synergistic when the SI was >1.

Combination with melphalan

Melphalan was purchased from Sigma Aldrich (catalog n°M2011). Combination between miR-17-92 LNA gapmer_06 and melphalan was tested in AMO1 cells. Cell number at plating was 1×10³ in 96-well plates. Melphalan was used at concentrations ranging from 0.5 μM to 2 μM. Measurement of synergistic index (SI) was determined with the following formula: SI=(effect induced by miR-17-92 LNA gapmers and melphalan in combination)/(effect of miR-17-92 LNA gapmers)+(effect of melphalan). Interactions were considered synergistic when the SI was >1.

Survival Assay

Cells were seeded in 100 μL (suspension cell lines) or 50 μL (adherent cell lines) of culture medium at a density ranging from 0,5 to 2,5×10³ cells per well. Survival was evaluated by both Cell Counting Kit-8 colorimetric assay (CCK-8, Dojindo Molecular Technologies, Japan) and CellTiter-Glo Luminescent Cell Viability Assay (Promega Madison, Wis., USA). Assays were performed according to the manufacturer's instructions. The optical density (OD) was evaluated at wave length of 450 nm. Both OD and luminescence were recorded with the GloMax-multi detection system (Promega, Madison, Wis., USA).

Flow Cytometry

Surface marker and cell viability analysis were performed using a FACSCalibur flow cytometer (Becton Dickinson, Franklin Lakes, N.J.). Phicoeritrin conjugated CD138 mAb (CD138-PE; Imgenex, San Diego, Calif.) was used to evaluate surface expression of CD138. 7-aminoactinomycin was used to evaluate cell viability. The staining was performed according to manufacturer's instructions. Data were analysed by flowing software (PertuuTerho, Centre for Biotechnology, Turku, Finland).

Reverse Transcription (RT) and Quantitative Real-Time Amplification (qRT-PCR)

Reverse transcription (RT) of both primary and mature miRNAs was performed using TaqMan® MicroRNA Assays, following a procedure for multiplexing the RT step (Life Technologies, Publication Part Number 4465407). Briefly, 500 ng of total RNA, prepared with the TRIzol® Reagent (Invitrogen), underwent reverse transcription by the Taq-Man® MicroRNA RT Kit. In this reaction, the RT primer pool was composed by the individual RT primers for each microRNA belonging to the miR-17-92 cluster and two RT primers to allow the reverse transcription of the miR-17-92 primary miRNA. Real-time PCR was performed using TaqMan® MicroRNA Assays together with the TaqMan® Universal PCR Master Mix on an ViiA7 System (Life Technologies). Both primary and mature microRNAs expressions were relatively quantified using the 2-ΔΔCt method (Applied Biosystems User Bulletin No. 2), and expressed as the relative quantity of target miRNA normalized to the RNU44 (assay ID 001094) housekeeping gene. Comparative real-time polymerase chain reaction (RT-PCR) was performed in triplicate, including no-template controls. Relative expression was calculated using the comparative cross threshold (Ct) method.

Immunoblotting Analysis

SDS-PAGE and Western Blotting (WB) were performed according to standard protocols. Briefly, cells were lysed by lysis buffer containing 15mM Tris/HCl pH 7.5, 120mM NaCl, 25mM KCI, 1mM EDTA, 0.5% Triton 100, Halt Protease Inhibitor Single-Use cocktail (100X, Thermo Scientific). Whole cells lysates (50 μg per lane) were separated using 4-12% Novex Bis-Tris SDS-acrylamide gels (Invitrogen), electro-transferred on Nitrocellulose membranes (Bio-Rad), and immunoblotted with the antibodies. Membranes were washed 3 times in PBS-Tween and then incubated with a secondary antibody conjugated with horseradish peroxidase in 0.5% milk for 2 hours at room temperature. Chemiluminescence was detected using Western Blotting Luminol Reagent (sc-2048, Santa Cruz, Dallas, Tex., USA). Signal intensity was quantified with the Quantity One Analyzing System (Bio-Rad).

Antibodies: PTEN (#9188), pan-AKT (#4691), phospho-AKT (#13038), p42-44 (tERK) (#4695), phospho-p42/44 (pERK) (#4370), p14/ARF (#2407), phospho-MDM2 (#3521), Puma (#12450), Bax (#5023), Bim (#2933), Bak (#12105), Bid (#2006), Mcl1 (#5453), Bcl2 (#2870), Caspase-3 (#9665) and GAPDH (#5174) were purchased from Cell Signalling Biotechnology; E2F-1 (sc-193) and p53 (sc-126) were purchased from Santa Cruz Biotechnology.

Animals and in vivo Model of Human MM.

Male CB-17 severe combined immunodeficient (SCID) mice (6- to 8-weeks old; Harlan Laboratories, Inc., Indianapolis) were housed and monitored in our Animal Research Facility. All experimental procedures and protocols had been approved by the Institutional Ethical Committee (Magna Graecia University) and conducted according to protocols approved by the National Directorate of Veterinary Services (Italy). In accordance with institutional guidelines, mice were sacrificed when tumors reached 2 cm in diameter or in the event of paralysis or major compromise, to prevent unnecessary suffering. Animal experimental procedures have been performed as in previous reports (Di Martino et al, CCR 2012; Di Martino el al. Oncotarget 2013, Di Martino et al PlosOne 2014). Briefly, mice were subcutaneously inoculated with 5×10⁶ NCI-H929 cells and treatments started when palpable tumors became detectable, approximately 3 weeks following injection of MM cells. Treatments were performed intraperitoneally with different schedules in order to reach the Maximun Tolerated Dose (MTD) and a dose-response curve. Mice were also inoculated with 5×10⁶ luciferase gene-marked U266, AMO-1, AMO-1/abzb, treatments were performed intraperitoneally (i.p.), and tumor volume were measured by IVIS Lumina II.

The SCID-hu model was performed implanting human fetal long bone grafts sc into SCID mice (SCID-hu). Four weeks following bone implantation, 2.5×10⁶ INA-6 multiple myeloma cells were injected directly into the human bone implant. Because INA-6 cells release soluble human IL-6 receptor (shuIL-6R), we used this marker to monitor tumor growth in SCID-hu mice.

The tumor sizes were assessed by Caliper measurement. Tumors were then collected and placed in either 10% formalin for histology analysis or, in RNAlater for RNA isolation or stored at −80° C. for protein analysis.

Quantification of IL-6 Production

Mouse sera were serially monitored for shulL-6R levels by ELISA (Quantikine Rat IL-6; R&D Systems). Mice developed detectable serum shuIL-6R ˜4 weeks following INA-6 cell injection and then were treated with. Three days after the last injection, blood samples were collected and analyzed (R&D Systems).

Statistical Analysis

All in vitro experiments were repeated at least 3 times and performed in triplicate; a representative experiment was showed in figures. Statistical significances of differences were determined using Student's t test, with minimal level of significance specified as P <0.05. Statistical significance of the in vivo growth inhibition observed in miR-17-92 LNA gapmer_06-treated mice compared with control group was determined using Student's t test. The minimal level of significance was specified as P <0.05. All statistical analyses were determined using GraphPad software (www.graphpad.com). Graphs were obtained using Microsoft Office Excel tool.

Pilot Toxicity Study in Cynomolgus Monkeys Captive-bred non-naïve cynomolgus monkeys were selected for entry into this study. The animals were allowed to acclimatize to the LPT (Laboratory of Pharmacology and Toxicology GmbH & Co. KG, Hamburg) primate toxicology accommodation for a period of 4 weeks before the commencement of the study. All animals were dewormed by LPT. The animals were selected for entry into the study based on the results of a satisfactory preliminary health screening. The route of administration was intravenous bolus injection into the vena cephalica of the left or right arm with the treatment on days 1, 4, 8, 15 and 22. The control group was treated with saline while three groups were treated with increasing doses of the gapmer_06 molecule diluted in saline. The administration volume was 1 ml/kg.

### Example 1

Relative Expression of miR-17-92 Cluster in MM Cell Lines

By the use of a custom q-RT-PCR plate specifically designed by us to detect all six mature miR-17-92 members and the primary transcript by two different assays (pri-mir-17/18a/19a and pri-mir-19b1/20a/92a1), we quantified in a panel of 11 MM cell lines the levels of the different miRNAs and pri-miRs.

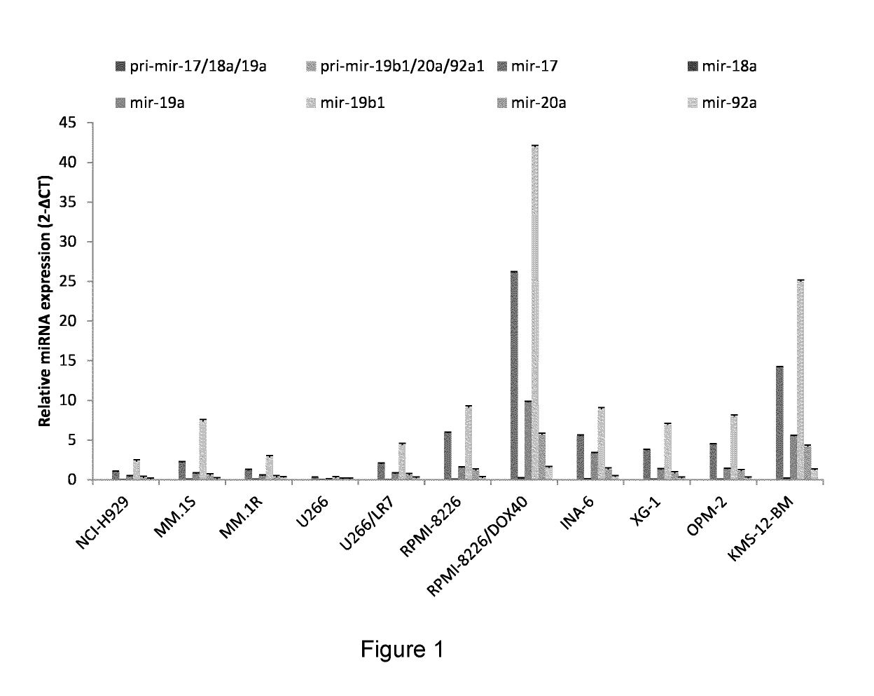

The results show miRNA expression in all cells (FIG. 1).

### Example 2

In vitro Screening of 8 miR-17-92 LNA/DNA Gapmers

Based on the sequence of the miR-17-92 primary transcript available on the NCBI web site (http://www.ncbi.nlm.nih.gov/nuccore/AB176708), we designed and explored eight different LNA/DNA gapmers oligonucleotides (Exiqon, Vedbaek, Denmark).

Names and sequences of the LNA/DNA gapmers are listed in Table 1 in the above description.

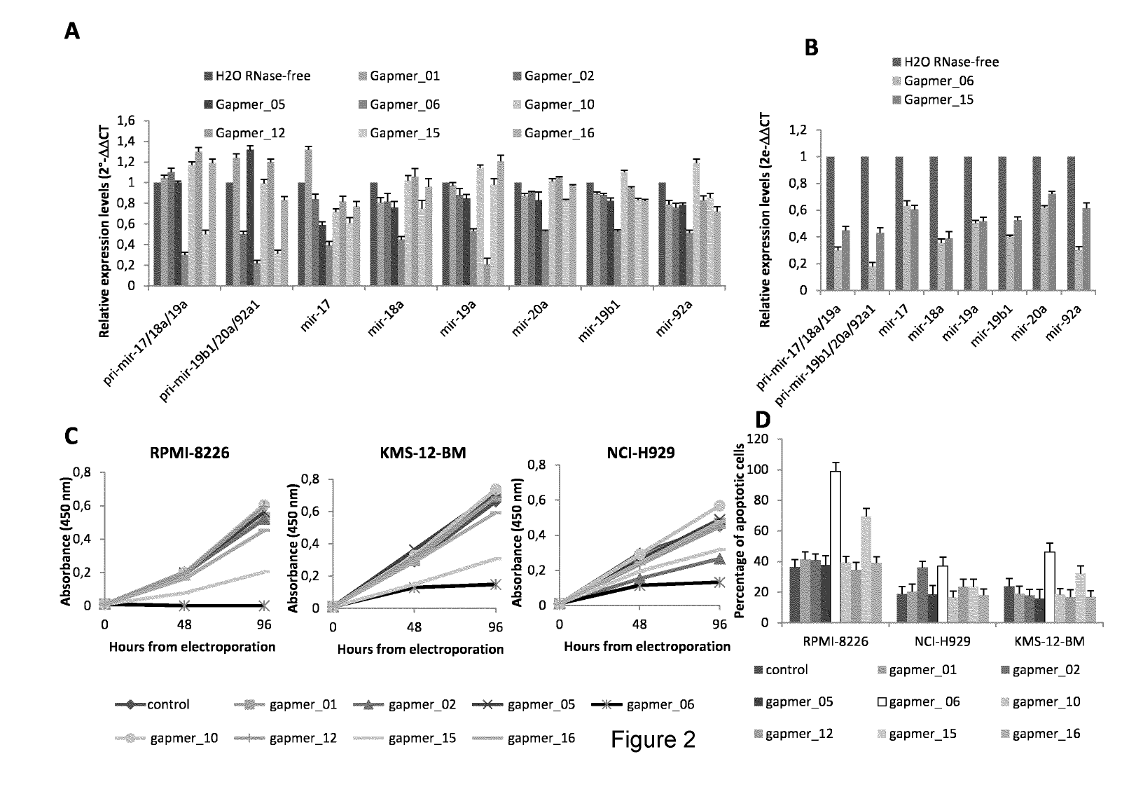

q-RT-PCR performed 48 hours after transfection of NCI-H929 and RPMI-8226 cells with 25 nM of each of the eight gapmer oligonucleotides, indicated that some gapmers downregulated single mature miRNAs, others downregulated also the pri-miRNAs. Among all the eight sequences the miR-17-92 LNA gapmer_06 is the more effective gapmer to downregulate both primary and mature miRNA sequences (FIG. 2A-B). Moreover, the miR-17-92 LNA gapmer_06 induced the strongest cell growth inhibition up to 96 hours after transfection, as assessed by CCK-8 assay (FIG. 2C) and increased apoptosis as assessed by AnnexinV/7-AAD staining (FIG. 2D) in RPMI-8226, KMS-12-BM and NCI-H929 MM cells.

All together, these results show that the oligonucleotide named miR-17-92 gapmer_06 [SEQ ID NO. 4] (black line/white bar), among the others, is the most efficient in targeting the pri-miR-17-92 and, consequently, in down-regulating the levels of mature miRNAs. Furthermore, this inhibition translates in the strongest growth inhibition and apoptosis induction.

### Example 3

Balance Between Antisense/Off-Target Activity of LNA Gapmers in MM Cells

Antisense oligonucleotides may retain off-target activities, other than the antisense activity. This is especially true for the new generation compounds, such as LNA/DNA gapmers, due to the chemical modifications carried out to enhance their stability and target affinity.

In order to assess the balance between antisense/off target effect of the selected LNA/DNA gapmer (gapmer_06; SEQ ID NO.4), we designed and tested four different experimental controls:

1) Scrambled LNA/DNA gapmers are generic scrambled oligonucleotides designed to assess whether MM cells are sensitive to gapmer compounds;

2) Mismatched LNA gapmer_06, are oligonucleotides with the same core DNA sequence flanked by mismatched LNAs at the ends, designed to rule out a sequence-specific toxicity of our LNA gapmers;

3) No-phosphorothioated (NP) LNA gapmer_06 were designed with the aim to exclude the off-target activity produced by the aspecific binding of phosphorothioated oligonucleotides to cellular proteins. NP gapmer_06 retain exclusively the antisense activity of the fully PS gapmer_06 (object of the present invention), obviously weakened by the lower stability and target affinity. These compounds have the meanings of positive experimental controls for antisense activity;

4) LNA mixmers_06, which include an additional LNA nucleotide within the DNA sequence, were designed to confirm the prominence assumed by the recruitment of RNase H in targeting pri-miRNAs. Moreover, LNA mixmers_06 serves as further control in ruling out a sequence-specific toxicity of our LNA/DNA gapmers.

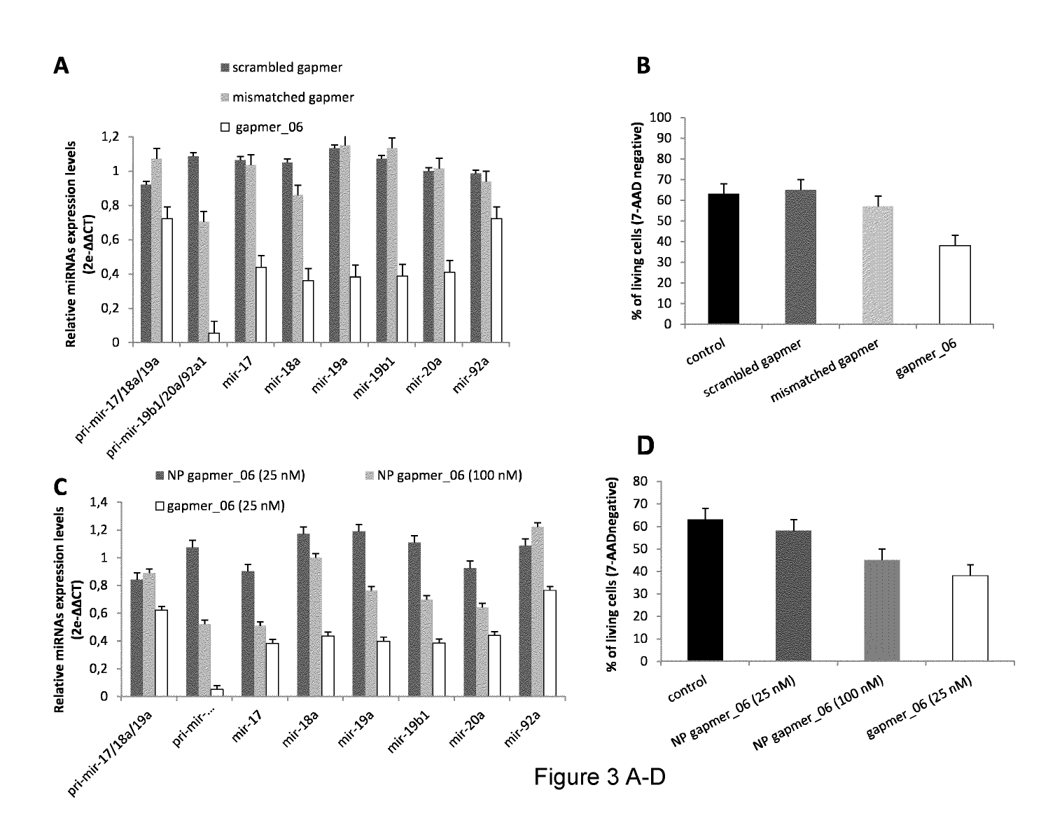

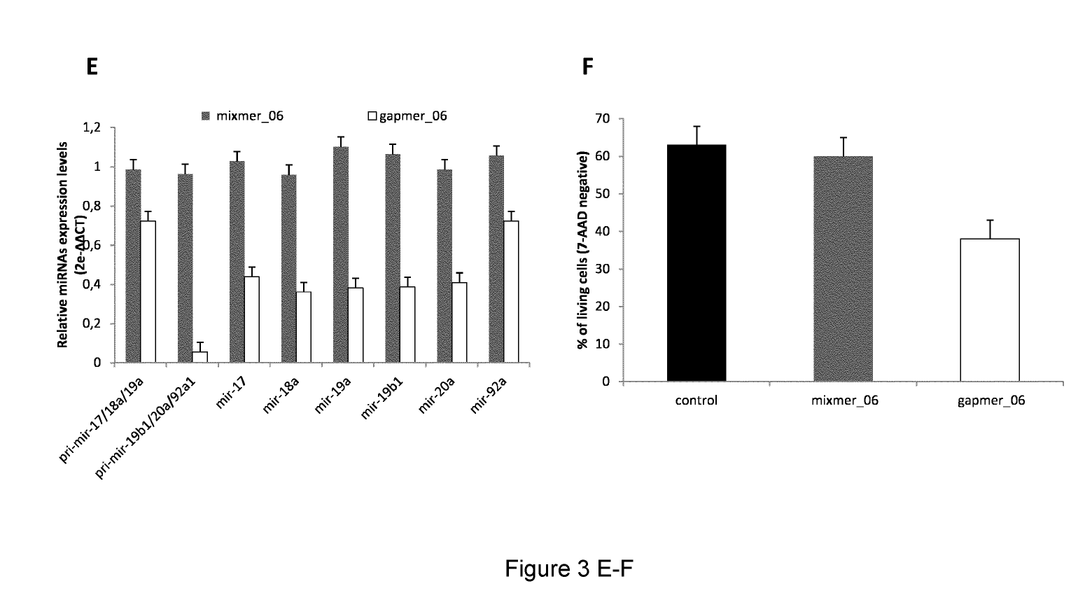

We transfected NCI-H929 cells with the four different experimental controls (scrambled gapmers_06, mismatched gapmers_06, NP gapmers_06 and mixmers_06). As shown in FIG. 3 frame A, neither transfection with scrambled gapmers_06 or transfection with mismatched gapmers_06 were able to downregulate pri-miRs and miR-17-92 cluster members. Both the scrambled and mismatched gapmers did not affected cell survival, as assessed by 7-AAD staining (FIG. 3 frame B). As shown in FIG. 3 frame C, transfection with NP gapmers_06 required higher concentrations (100 nM versus 25 nM) to exert the antisense activity, as compared to the fully PS gapmers_06. Importantly, transfection with 100 nM of NP gapmers_06 downregulated pri-miRs and miR-17-92 cluster members and resulted in induction of cell death (FIG. 3 frame D). As shown in FIG. 3 frame E, transfection with mixers_06 failed to downregulate pri-miRs and miR-17-92 cluster members. Moreover viability of NCI-H929 cells was not affected by mixmers_06 (FIG. 3 frame F).

All together, these results confirm that gapmer_06 is a specific inhibitor of the miR-17-92 cluster of microRNAs and that the observed anti-MM activity specifically correlate with the antisense activity.

### Example 4

Antiproliferative Activity of miR-17-92 Gapmer_06 in Different MM Cell Lines

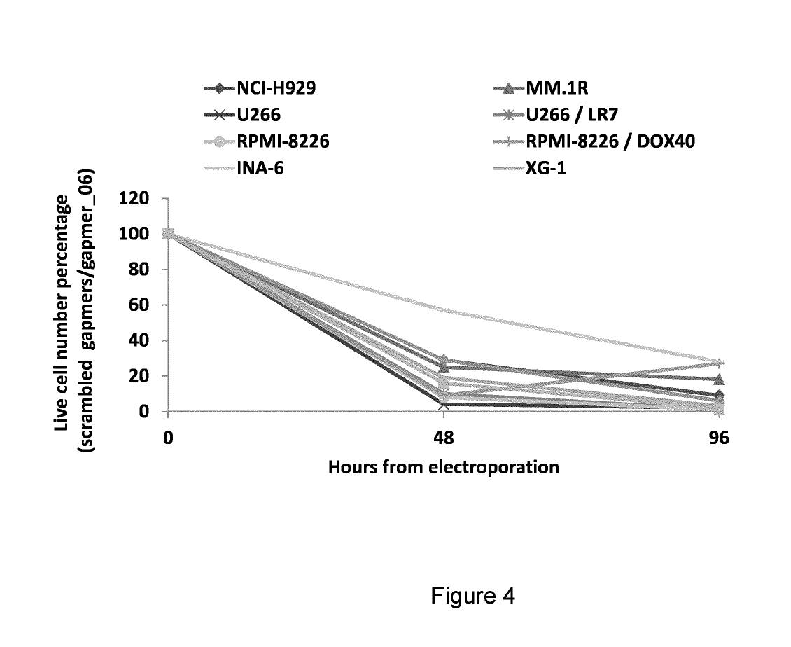

We evaluated effects induced on proliferation in a panel of MM cell lines by miR-17-92 gapmer_06 transfection. As assessed by CCK-8 assay, our selected gapmer exerted anti-proliferative activity up to 96 hours after transfection in all myeloma cell lines tested (FIG. 4).

### Example 5

Myeloma Cells are More Sensitive to Gapmer_06 with Respect to Mature miRNA Inhibitors

We evaluated whether the targeting of the pri-miR-17-92 by LNA gapmers was more effective against myeloma cells as compared with the targeting of the single miR-17-92 mature miRNAs. To this aim, we transfected three different MM cell lines with 25 nM of either gapmer_06 or miRNA inhibitors from Ambion or RNase free water as control.

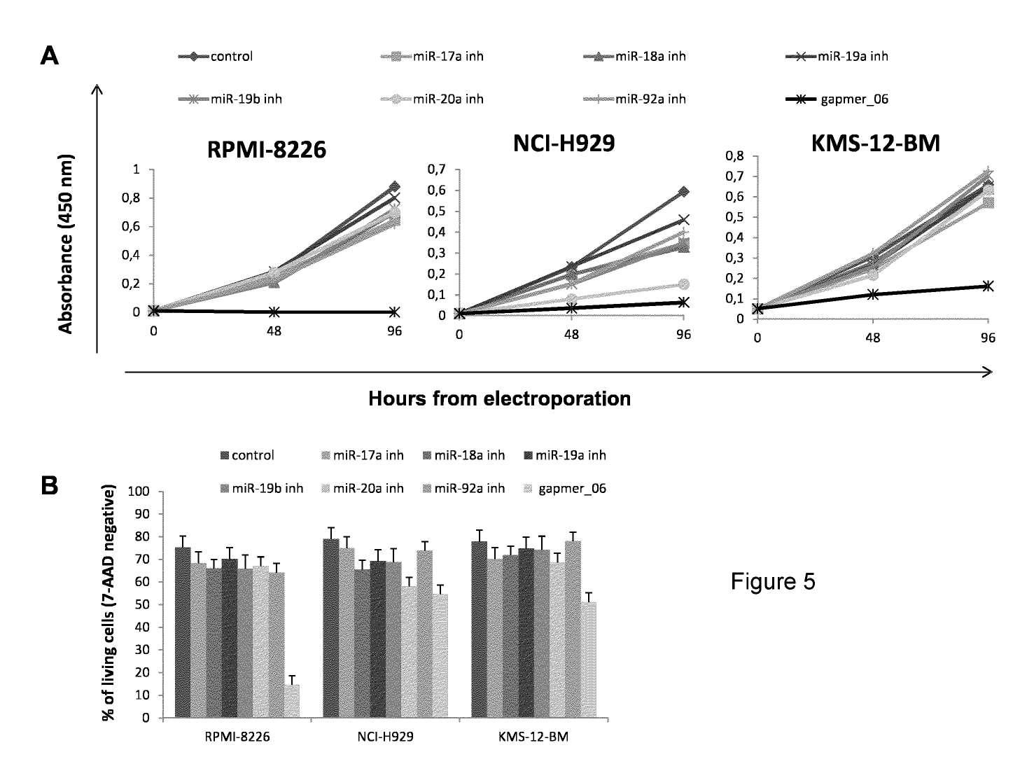

As shown in FIG. 5, the strongest biological effects, as assessed by CCK-8 assay (A) and flow cytometry analysis (B), were induced by the LNA/DNA gapmer (black line in A and first column on the right in B) in all MM cell lines tested.

Overall, these findings suggested that targeting the pri-miRNA is a more useful approach to affect MM cell growth and survival as compared to targeting single mature miRNAs.

### Example 6

MM Cells are Sensitive to Gymnotic Exposure to LNA Gapmers

Gymnotic delivery of naked oligonucleotides takes advantage of the normal growth properties of cells in tissue culture in order to promote productive oligonucleotides uptake. Up to now, the optimum results were obtained with LNA/DNA gapmers (Stein C.A. et al NAR 2010).

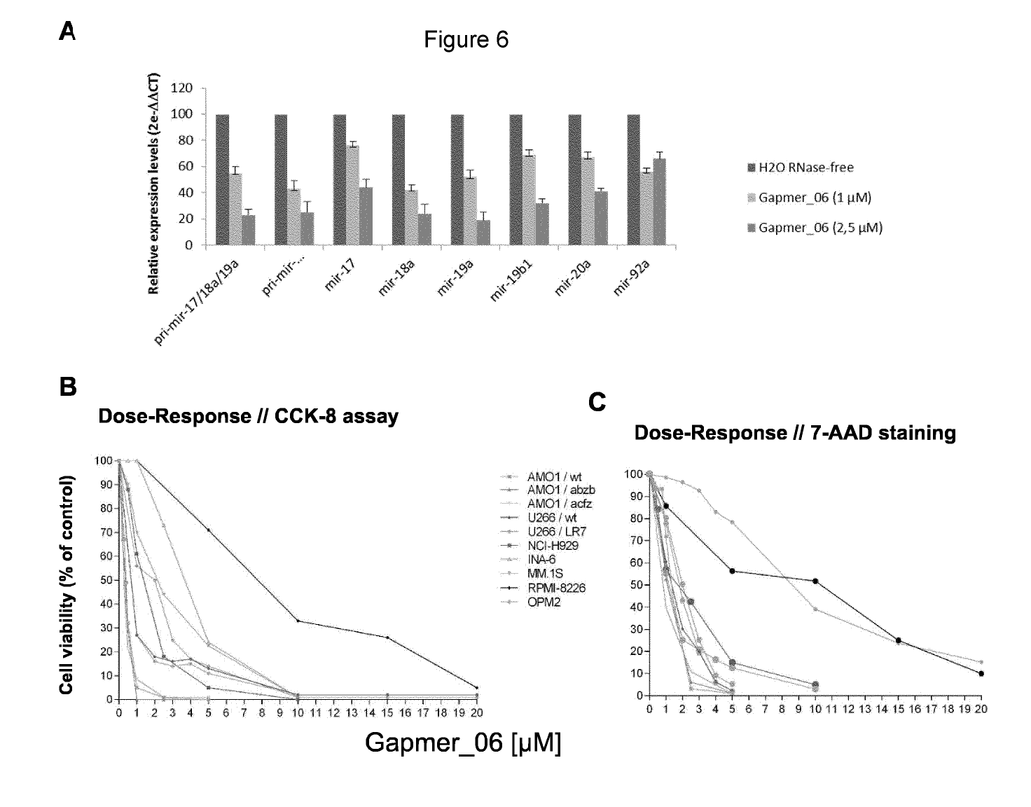

Here, we tested if gymnotic treatment can efficiently deliver our LNA/DNA gapmers to myeloma cells. By q-RT-PCR we evaluated expression levels of both pri- and mature miRNAs in two MM cell lines after six days of gapmer_06 exposure. We found in both cell lines downregulation of primary transcript as well as mature single miRNAs, in a dose dependent manner, as compared to saline vehicle alone (FIG. 6—saline vehicle not shown).

In order to evaluate the sensitivity of MM cells to LNA/DNA gapmers, we exposed for six days ten MM cell lines to concentrations of gapmer_06 ranging from 100 nM to 20 μM. As shown in FIG. 6, frames B and C, MM cells exhibit different sensitivity to LNA gapmers. By CCK-8 assay we detected IC₅₀ values ranging from 500 nM to about 8 μM, while by flow cytometry analysis of 7-AAD stained cells we calculated IC₅₀ values ranging from 1.25 μM to about 11 μM. Taken together, these data demonstrate that 1) LNA/DNA gapmer is efficiently gymnotically uptaken from MM cells; 2) All MM cell lines are sensitive to gymnotic exposure to LNA gapmers.

### Example 7 Exposure to LNA/DNA Gapmers Affects Survival of Primary MM Cells

CD138+malignant plasma cells derived from bone marrow aspirates of 11 myeloma patients were cultured in presence of human stromal HS-5 cells.

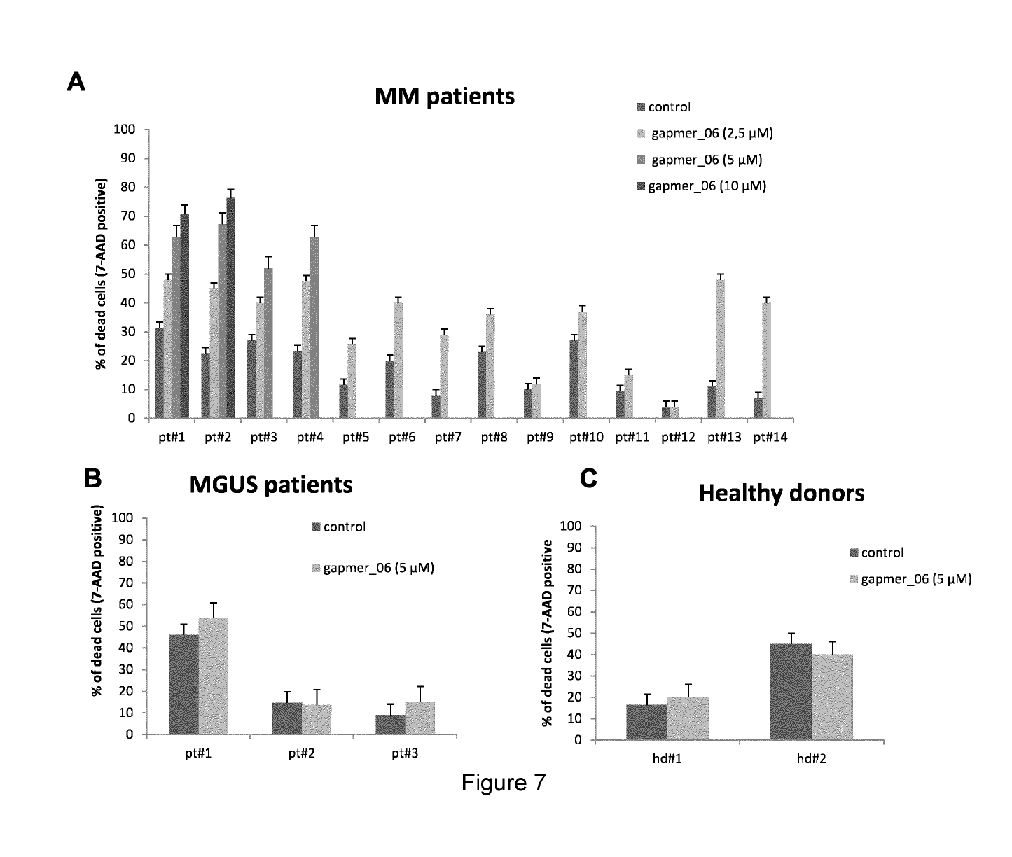

After 6 days of gymnotic exposure to gapmer_06 (2.5, 5 or 10 μM), we detected a significant increase of dead myeloma cells, as assessed by 7-AAD staining (FIG. 7A).

In contrast CD138+plasma cells derived from MGUS patients or PBMCs from healthy donors were unaffected from the exposure to the gapmer_06 (FIG. 7B-C), indicating a specific sensitivity of malignant plasma cells to inhibition of miR-17-92 cluster.

### Example 8

Synergistic Induction of Cell Death by miR-17-92 Gapmer_06 and Conventional Anti-MM Agents

Proteasome inhibitors as well as melphalan actually constitute the mainstay treatment for MM patients. Here, we investigated the effects produced by combination treatment of MM cells with LNA/DNA gapmers and the proteasome inhibitors including bortezomib and carfilzomib, or melphalan.

AMO1 cells were exposed for 6 days to LNA gapmers_06 or each of Bortezomib, Carfilzomib, Melphalan or to combinations of LNA gapmer_06 and each of these drugs.

As shown in the following Tables 2-4 LNA gapmer_06 synergizes with proteasome inhibitors Bortezomib and Carfilzomib, and with Melphalan to induce cell death in AMO1 cell line.

Cells viability was calculated by flow cytometric analysis of 7-AAD staining and the percentage of living cell was compared to the untreated cells.

TABLE 3
CI value of combination of different concentrations of LNA gapmer_06
with Melphalan. (CI < 1 synergism; CI = 1 additive; CI > 1 antagonism)

| | miR-17-92 LNA gapmer (μl) | | | |
|---|---|---|---|---|
| Melphala (μm) | 0.5 | 0.75 | 1 | 1.25 |
| 1.5 | 0.78 | 0.99 | 0.65 | 0.56 |
| 1.75 | 0.66 | 0.89 | 0.56 | 0.47 |
| 2 | 0.51 | 0.59 | 0.46 | 0.26 |

TABLE 4
CI value of combination of different concentrations of LNA gapmer_06
with Bortezomib. (CI < 1 synergism; CI = 1 additive; CI > 1 antagonism)

| | miR-17-92 LNA gapmer (μl) | | | |
|---|---|---|---|---|
| Bortezomib (μm) | 0.5 | 0.75 | 1 | 1.25 |
| 0.0035 | 0.759 | 0.418 | 0.342 | 0.036 |

TABLE 5
CI value of combination of different concentrations of LNA gapmer_06
with Carfilzomib. (CI < 1 synergism; CI = 1 additive; CI > 1 antagonism)

| | miR-17-92 LNA gapmer (μl) |
|---|---|
| Carfilzomib (μm) | 1.25 |
| 0.0004 | 0.89 |
| 0.0005 | 0.55 |
| 0.0006 | 0.50 |
| 0.0007 | 0.39 |

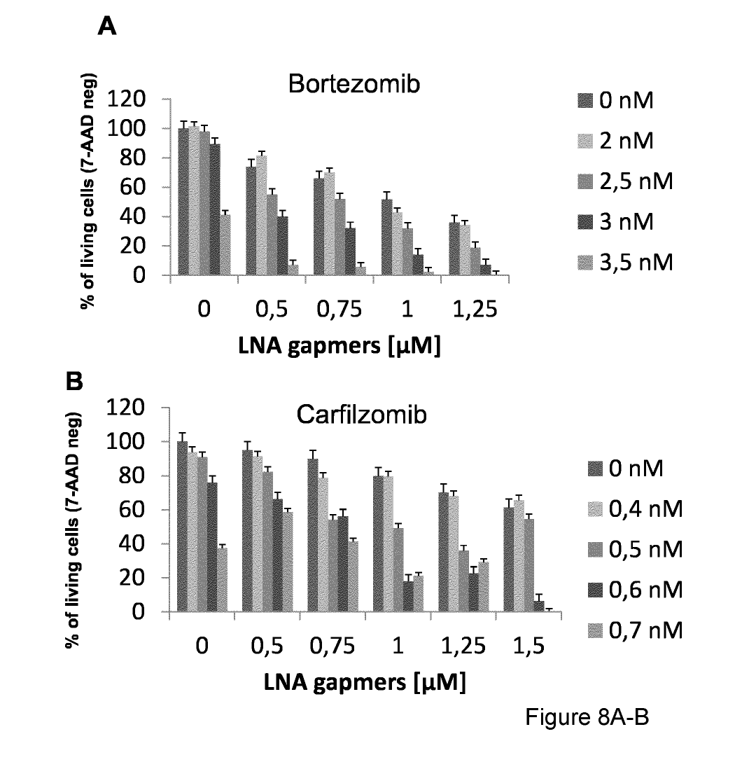

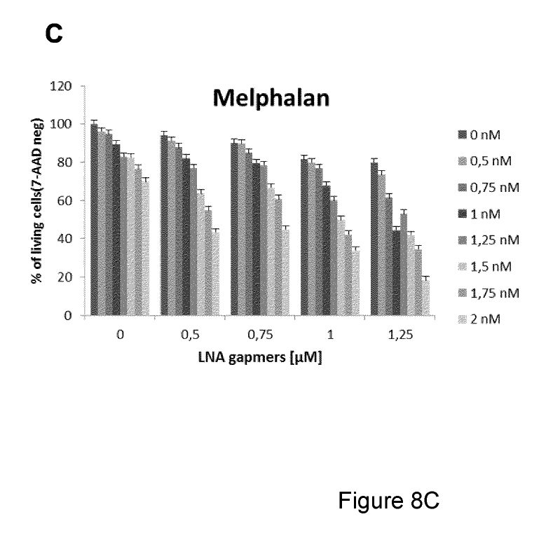

In Tables 3-5 tests with the respective combinations performed simultaneously are reported. The effect of LNA gapmer_06 was determined separately for Bortezomib, Carfilzomib, and Melphalan explaining the slightly different values. CI (combination index) values are shown (CI<1 synergism, CI=1 additive, CI<1 antagonism). For each monotherapy different doses and combinations were tested. Combination index were calculated using the CalcuSyn software. As shown in FIG. 8 frames A, B and C, gapmer_06 and proteasome inhibitors (Bortezomib and Carfilzomib) or melphalan synergically affect AMO1 cells survival, as assessed by flow cytometric analysis of 7-AAD stained cells.

This finding strongly supports the translational relevance of our invention in a combination regimen with conventional anti-MM agents.

### Example 9

Molecular Perturbations Caused by miR-17-92 Gapmer_06 in myeloma Cells

MiRNAs are a class of non coding RNAs that regulate gene expression at post-transcriptional levels. Each miRNA may regulate the expression of several (hundreds) genes. Since LNA/DNA gapmers inhibit the biogenisis of the six members of the miR-17-92 cluster, a wide perturbation in the expression of target genes is predictable. Therefore, we gymnotically exposed NCI-H929 cells for 6 days to gapmer_06 or scrambled controls and, by western blotting, we analyzed molecular perturbations at protein levels.

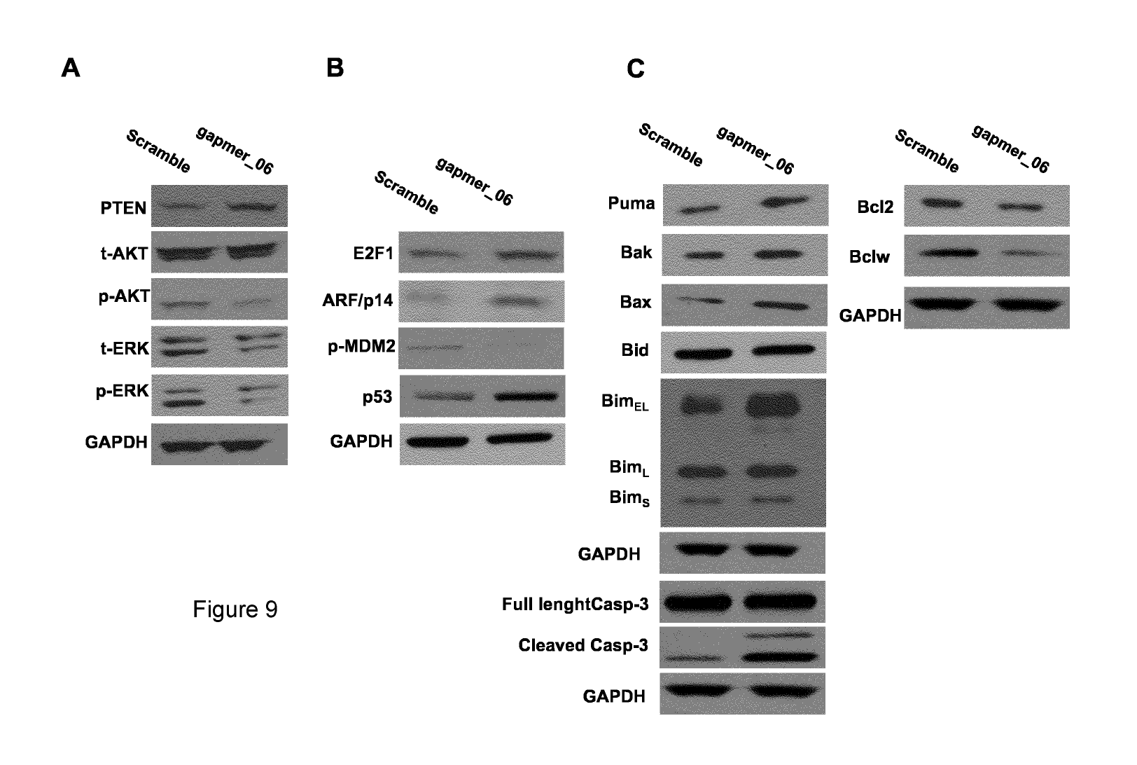

As shown in FIG. 9 frame A, exposure to gapmer_06 induces a strong up-regulation of PTEN (validated target of miR-17/19a/19b/20a/92a) that consequently impairs the activity of both AKT and ERK proteins, as demonstrated by the reduction of the phopshorylated / active forms (p-AKT and p-ERK).

As shown in FIG. 9 frame B, exposure to gapmer_06 triggers the p14/ARF-p53 fail-safe axis in the p53-wild-type NCI-H929 cells. Induction of this pathway is mediated by E2F-1, a validated target of both miR-17 and miR-20, which results strongly up-regulated after treatment with gapmer_06.

Finally, as shown in FIG. 9 frame C, exposure to gapmer_06 leads to the activation of the intrinsic apoptotic pathway, characterized by the up-regulation of pro-apototic proteins (left; Puma, Bak, Bax, Bid and Bim) and the down-regulation of anti-apoptotic proteins (right; Bcl-2, Mcl-1). Activation of this signaling results in caspase-3 cleavege and cellular apoptosis.

### Example 10

In vivo Anti-MM of miR-17-92 Gapmer_06

We next investigated the effect of gapmer_06 treatment against MM xenografts in SCID.NOD mice. When NCI-H929 MM tumors became palpable, mice were randomized in 6 different groups, to receive intraperitoneally: saline solution, gapmer_06 at concentration of 75-50-25 μg once weekly of or 25-12.5 μg twice weekly.

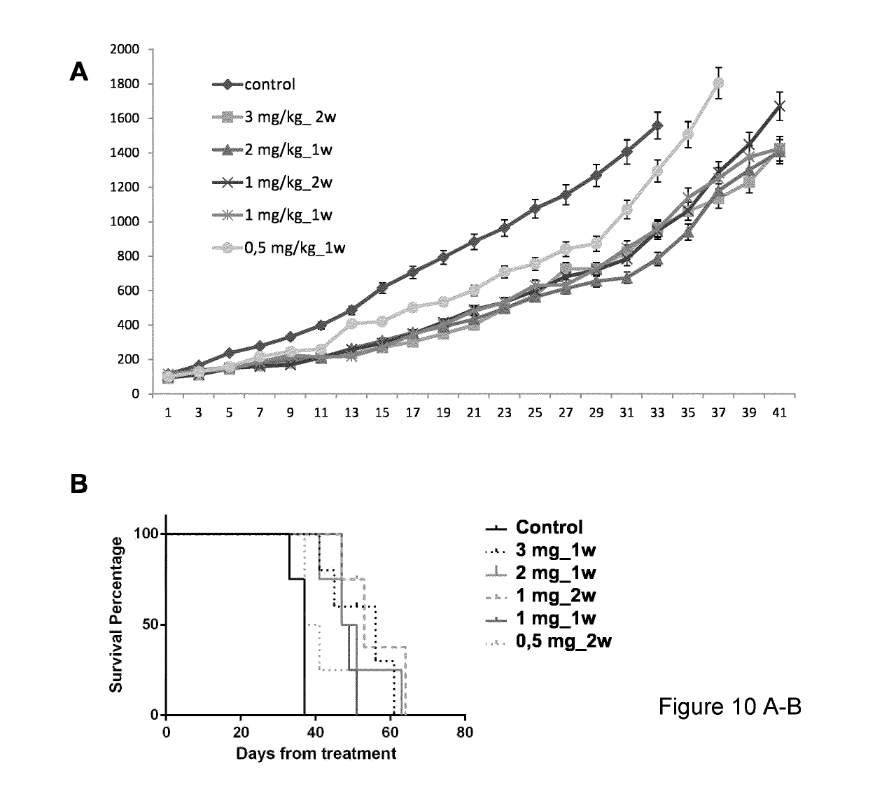

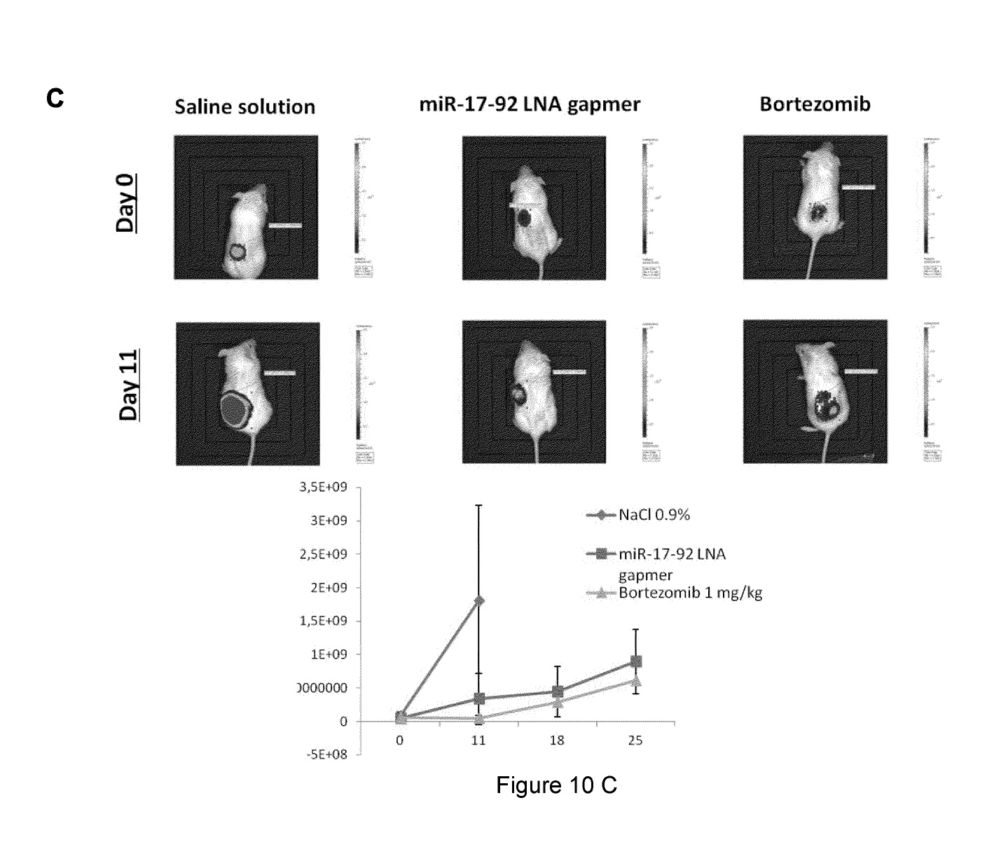

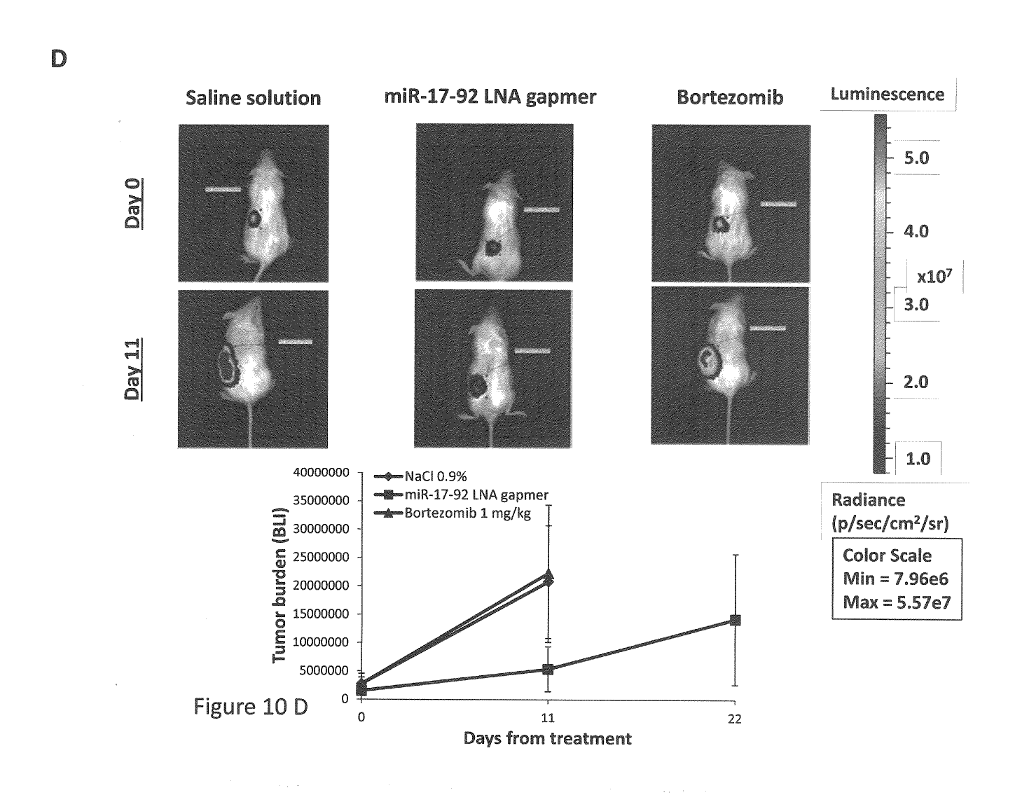

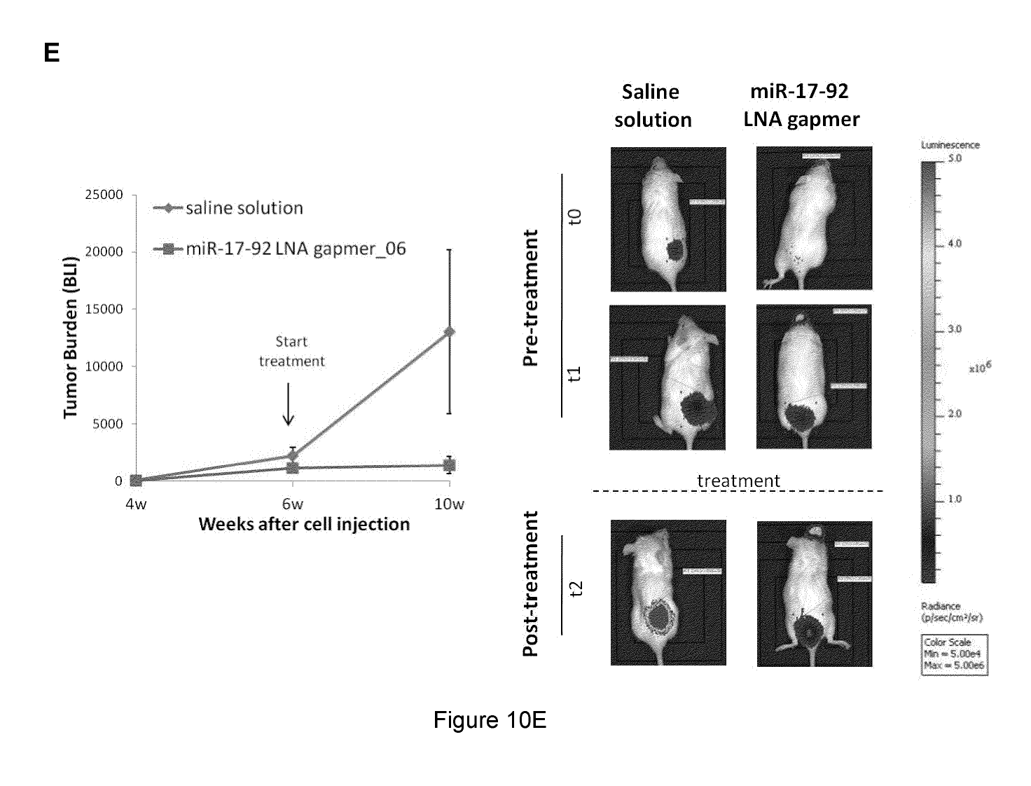

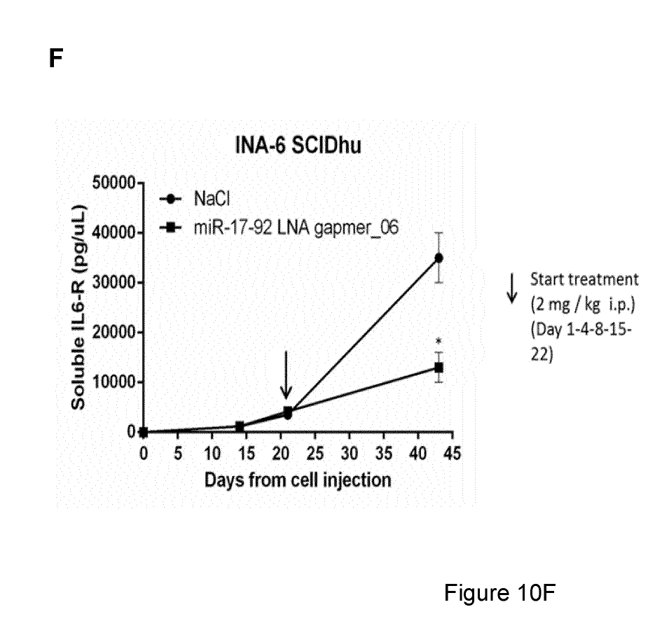

As shown in FIG. 10 frame A, gapmer_06 induced a significant (P<0.05) inhibition of tumor growth in a dose dependent-manner, reaching a plateau at the dose regimens of 25 μg twice weekly and 75-50 μg once weekly.

Survival analysis was performed and in FIG. 10 frame B is reported the Kaplan-Meier curve of gapmer_06 treated mice. Importantly, treatment with gapmer_06 resulted in a significant prolongation of survival (P<0.05), as compared to treatment with saline. All together, our results indicated an in vivo anti-MM activity of gapmer_06.

In vivo anti-MM activity of LNA gapmer_06 was next evaluated in NOD/SCID mice bearing subcutaneous AMO1 xenografts. In this model, luciferase gene-marked AMO1 xenografts, sensitive to Bortezomib, were I.P. treated with 2 mg/kg G06 and with Bortezomib 1 mg/kg at day 1-4-8-15-22. As shown in FIG. 10 C treatment with LNA gapmer_06 resulted in a significant tumor-growth inhibition. Importantly, effect of LNA gapmer_06 treatment is almost comparable to Bortezomib effect on tumor-growth inhibition. Tumors volume were measured using IVIS LUMINA II Imaging We next evaluate the in vivo activity in Bortezomib-refractory model of human MM. NOD/SCID mice bearing subcutaneous AMO1/abzb (Bortezomib-refractory cell line, luciferase gene-marked) xenografts, were I.P. treated with LNA gapmer_06 2 mg/kg and with Bortezomib 1 mg/kg. As shown in FIG. 10D treatment with LNA gapmer_06 resulted in a significant tumor-growth inhibition, compared to treatment with Bortezomib that have no effect on tumor-growth. Tumors volume were measured using IVIS LUMINA II Imaging System.

The in vivo activity of gapmer_06 was also evaluate in a orthotopic model of human MM using U266 cells, to explore the efficacy in relation to the microenvironment. Orthotopic murine model was performed by the use of NOD/SCID mice intravenously injected with luciferase gene-marked U266 cells. Tumor growth was monitored using IVIS LUMINA II Imaging System. Mice were I.P. treated with saline solution (control) and with LNA gapmer_06 2 mg/kg at day 1-4-8-15-22. Engraftment was determined by sacrificing mice after 10 weeks and analyzing bone marrow for luciferase gene-marked U266 cells. As shown in FIG. 10E, after LNA gapmer_06 treatment it was observed a significant tumor-growth inhibition compared to controls. Finally, we performed an INA-6 SCID-hu model to evaluate the activity of gapmer_06 on MM cells in the human bone marrow milieu. To this aim, a murine model of human MM was used, in which INA-6 cells are directly injected and engrafted into a human fetal bone chip implanted in a SCID mouse (SCID-hu mouse). This model allows the growth of human IL-6—dependent MM cells in a human bone marrow microenvironment. Mice were treated I.P. with gapmer_06 at concentration of 2 mg/kg at day 1-4-8-15-22, and serum shulL-6R levels were serially determined as a marker of tumor growth. As shown in FIG. 10 F treatment with LNA gapmer_06 leads to a significant tumor-growth inhibition, compared to controls.

### Example 11

miR-17-92 Gapmer_06 Toxicity in vivo Evaluation

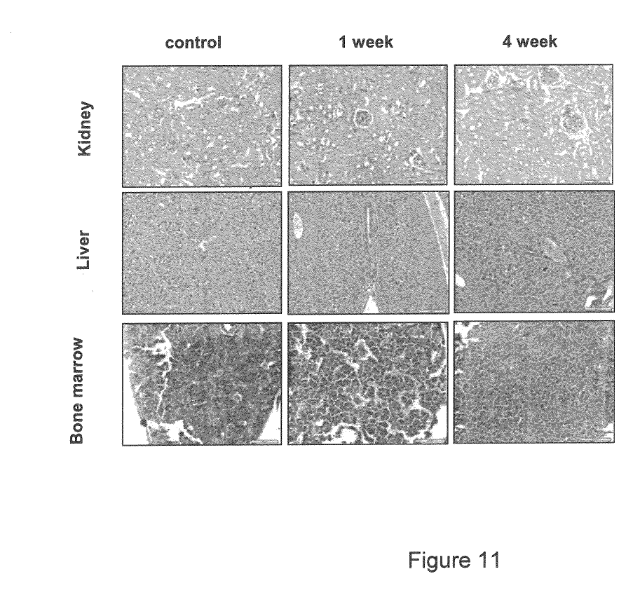

Hematoxylin and eosin staining (20-fold magnification) of kidney, liver and bone marrow retrieved from mice treated with 2 mg/kg of gapmer_06 or saline at days 1, 4, 7, 14 and 22. No significant damage was detected in the different groups of treatment. Representative images are shown (FIG. 11).

### Example 12

Exposure to gapmer_06 Affects Proliferation of Different Cancer Cell Lines in vitro

MiR-17-92 cluster is widely recognized as “oncomiR” in almost all tumor types. For this reason, we explored the activity of our gapmer-06 in cancer cell lines other than myeloma cells.

Specifically, we gymnotically exposed for 6 days to 0-2,5-5-10 μM of gapmer_06, the following cell lines: Capan1 pancreatic cancer cells, MCF7 and MDA231 breast cancer cells, LXF-289, A-549 and NCI-H82 lung cancer cells, 5637 bladder cancer cells, BCMW.1 Waldenstrom Macroglobulinemia cells and MPM-209 mesothelioma cells.

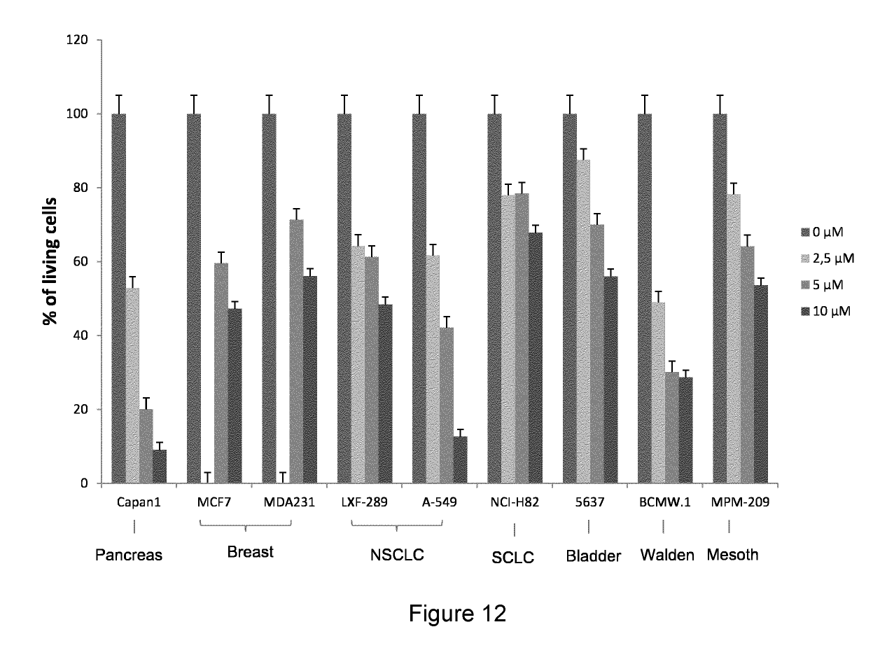

Cell growth analysis indicated that gapmer_6 impair proliferation of all cell lines (FIG. 12).

All together, these findings suggested anti-tumor activity of our miR-17-92 gapmer_06 in several human cancers and provide the rationale to extend the clinical translation beyond myeloma.

### Example 13

Gapmer_06 Pilot Toxicity Study in Cynomolgus Monkeys

Dose Regimen

| Group | | Dose level | Administration | Animal number |
|---|---|---|---|---|
| number | Treatment | mg/kg | number | and sex |
| 1 | saline | 0 | 5 | 1 female |
| 2 | gapmer_06 | Low (0.126) | 5 | 1 female |
| 3 | gapmer_06 | Mid (0.252) | 5 | 1 female |
| 4 | gapmer_06 | High (0.504) | 5 | 1 female |

Observations

| Parameters | Time points |
|---|---|
| Clinical | Daily. |
| signs/mortality | Observation include skin, eyes, mucous membranes, |
| | respiratory and circulatory systems, somatomotor |
| | activity and behavior patterns. Recording of the |
| | onset, intensity and duration of any observed signs, |
| | including tolerance at the injection site. |
| Body weight | Weekly |
| Food/water | Daily. Report include the weekly mean values |
| consumption | |
| Hematology, | All animal groups: |
| coagulation and | Prior to first administration (day −7) |
| biochemistry | Predose, days 4, 8, 15, 22 |

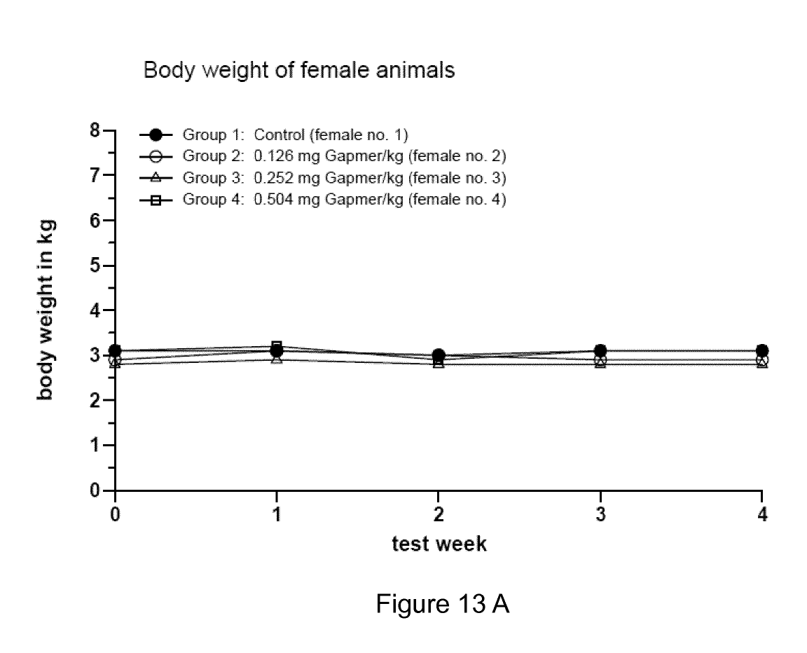

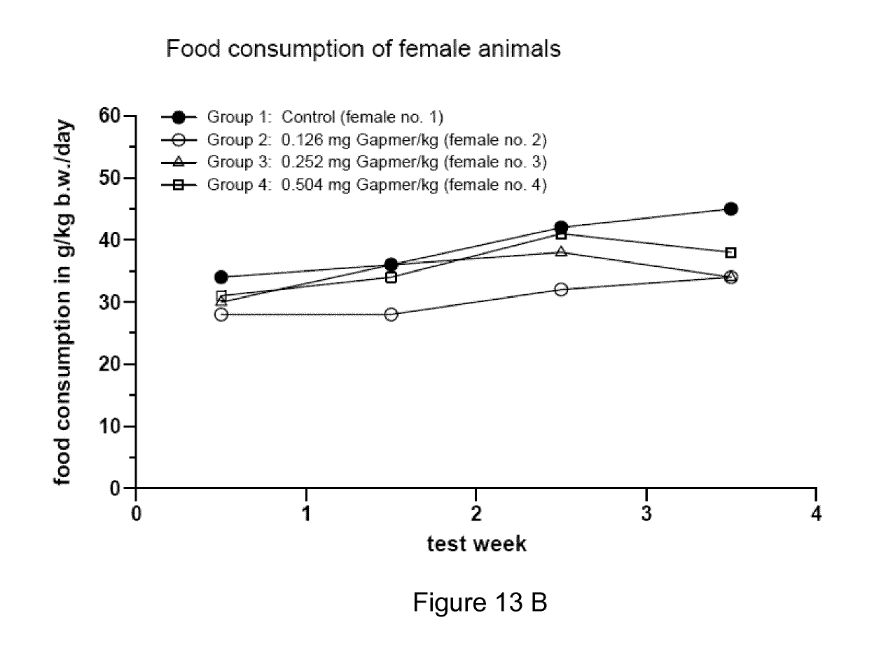

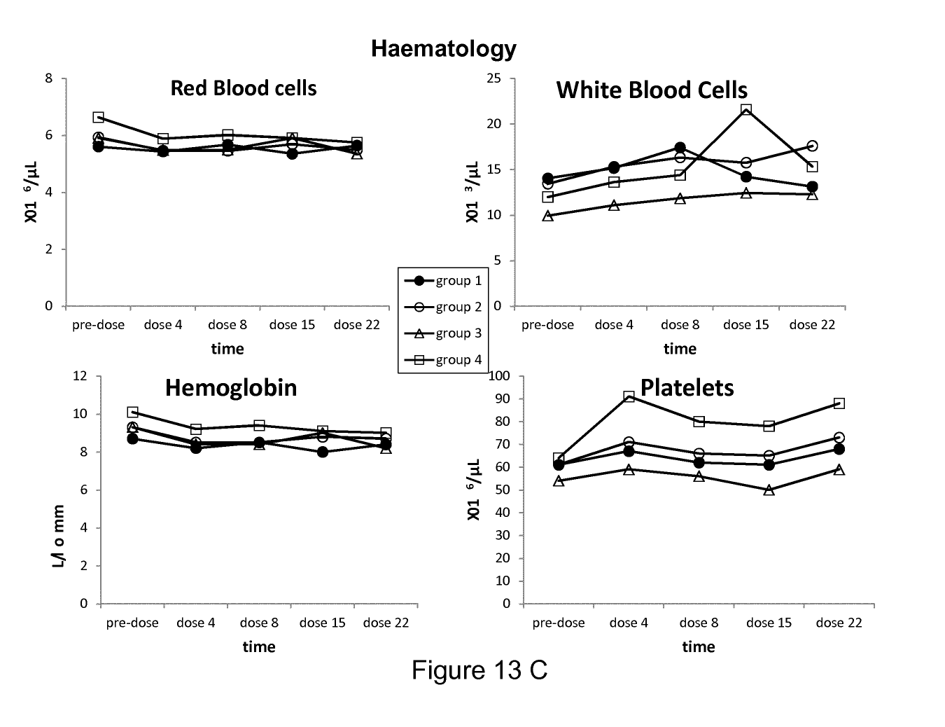

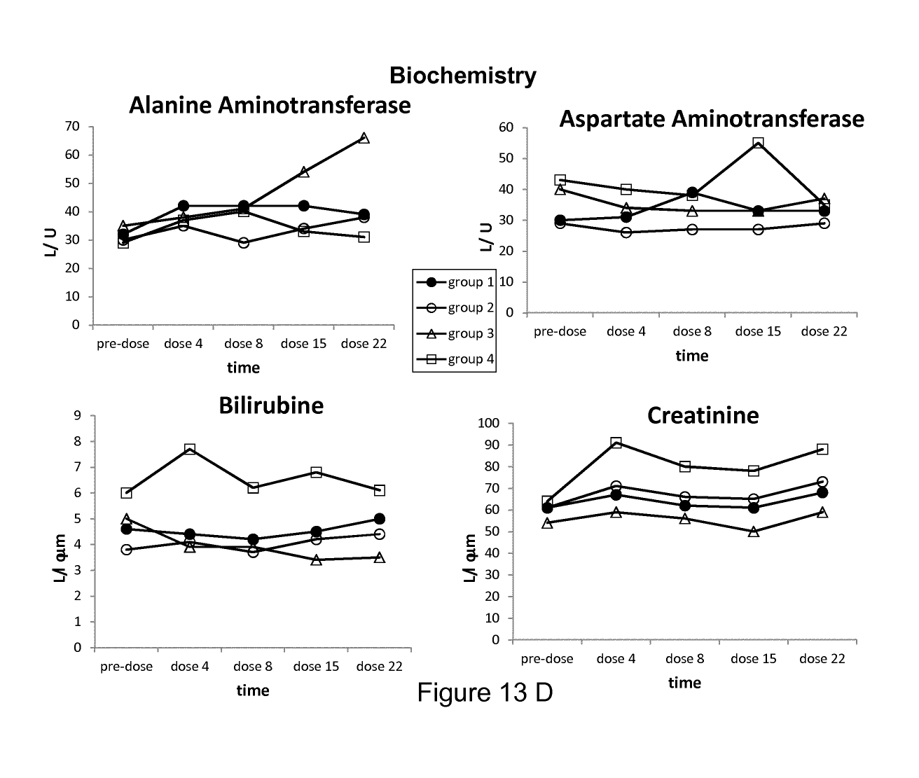

After completion of the administration period, no influence was noted on the behaviour, the body weight or the food consumption of the treated animals compared to the control group as reported in FIG. 13 A-B.

Further, the laboratory parameters appeared not to be influenced by the treatment with the three different dose levels of Gapmer_06. In FIG. 13 C-D the main haematology and biochemistry results are reported.

1. An LNA DNA gapmer which binds to a region of the primary miRNA (pri- miRNA) of the miR-17-92 cluster, said LNA/DNA gapmer having a nucleotide sequence selected from the group consisting of:
[SEQ ID NO. 4]
+T*+A*+C*T*T*G*C*T*T*G*G*+C*+T*+T,
[SEQ ID NO. 7]
+A*+G*+C*A*C*T*C*A*A*C*A*T*C*+A*+G*+C,
[SEQ ID NO. 1]
+C*+T*+G*T*A*A*G*C*A*C*T*T*T*+G*+A*+C,
[SEQ ID NO. 2]
+A*+C*+A*T*C*G*A*C*A*C*A*A*+T*+A*+A,
[SEQ ID NO. 3]
+T*+C*+A*G*T*A*A*C*A*G*G*A*C*+A*+G*+T,
[SEQ ID NO. 5]
+A*+T*+G*C*A*A*A*A*C*T*A*A*C*+A*+G*+A,
[SEQ ID NO. 6]
+G*+A*+A*G*G*A*A*A*T*A*G*C*A*+G*+G*+C,
and
[SEQ ID NO. 8]
+C*+G*+A*C*A*G*G*C*C*G*A*A*+G*+C*+T,
wherein letters with symbol “+” indicate the positions of LNA and symbol “*” indicates phosphorothioate bonds.

2. The LNA/DNA gapmer according to claim 1 for use as a medicament.

3. An inhibitor of the primary miRNA (pri-miRNA) of the miR-17-92 cluster for use in the treatment of tumors related to an overexpression of any of the miRNAs of the miR-17-92 cluster.

4. The inhibitor of claim 3, which is a LNA/DNA gapmer which specifically binds to a region of said pri-miRNA.

5. The inhibitor of claim 3, which is a LNA/DNA gapmer selected from the group consisting of sequences:
[SEQ ID NO. 4]
+T*+A*+C*T*T*G*C*T*T*G*G*+C*+T*+T,
[SEQ ID NO. 7]
+A*+G*+C*A*C*T*C*A*A*C*A*T*C*+A*+G*+C,
[SEQ ID NO. 1]
+C*+T*+G*T*A*A*G*C*A*C*T*T*T*+G*+A*+C,
[SEQ ID NO. 2]
+A*+C*+A*T*C*G*A*C*A*C*A*A*+T*+A*+A,
[SEQ ID NO. 3]
+T*+C*+A*G*T*A*A*C*A*G*G*A*C*+A*+G*+T,
[SEQ ID NO. 5]
+A*+T*+G*C*A*A*A*A*C*T*A*A*C*+A*+G*+A,
[SEQ ID NO. 6]
+G*+A*+A*G*G*A*A*A*T*A*G*C*A*+G*+G*+C,
and
[SEQ ID NO. 8]
+C*+G*+A*C*A*G*G*C*C*G*A*A*+G*+C*+T
wherein letters with symbol “+” indicate the positions of LNA and symbol “*” indicates phosphorothioate bonds.

6. The inhibitor of for of claim 3, wherein said tumor is selected from the group consisting of: multiple myeloma, Waldenstrom Macroglobulinemia, mesothelioma, bladder cancer, B-cell Lymphomas, B-cell Chronic Lymphocytic Leukemia, Acute Myeloid Leukemia, T-cell Lymphoma, Retinoblastoma, Osteosarcoma, Colorectal Cancer, Head and Neck Cancers, Pancreatic Cancer, Breast Cancer, Ovarian Cancer, Lung Cancer, Renal Cancer and Hepatocellular Carcinoma.

7. A pharmaceutical composition comprising one or more inhibitors of the primary miRNA of miR-17-92 cluster as active ingredients for use in the treatment of tumors related to an overexpression of any of the miRNAs of the miR-17-92 cluster.

8. The pharmaceutical composition for the use according to claim 7 wherein said tumor is selected from the group consisting of multiple myeloma, Waldenstrom Macroglobulinemia, pancreatic cancer, breast cancer, lung cancer, bladder cancer and pleural mesothelioma cancer.

9. A pharmaceutical composition comprising an inhibitor of the primary miRNA (pri-miRNA) of the miR-17-92 cluster and one or more proteasome inhibitors compound for use in the treatment of multiple myeloma.

10. A pharmaceutical composition comprising at least one of the LNA DNA gapmers of claim 1.

11. The pharmaceutical composition according to claim 10 further comprising a proteasome inhibitor compound.

12. The pharmaceutical composition according to claim 11 wherein said proteasome inhibitor is selected from bortezomib and carfilzomib.

13. The pharmaceutical composition according to claim 11 for use in the treatment of multiple myeloma.

14. A pharmaceutical composition comprising an inhibitor of the pri-miRNA of the miR-17-92 cluster and an active ingredient with anti-myeloma activity for use in the treatment of multiple myeloma.

15. A pharmaceutical composition comprising at least one of the LNA DNA gapmers of claim 1 and an active ingredient with anti-myeloma activity.

16. The pharmaceutical composition according to claim 15 wherein said active ingredient with anti-myeloma activity is melphalan.

17. The pharmaceutical composition according to claim 15 comprising the LNA DNA gapmer with SEQ ID NO. 4 (gapmer_06) and melphalan.
